SEQUENCE LISTING (62 sequences)

SEQ ID NO: 1 - DNA, 63 bp, Homo sapiens
  source 1..63: mol_type = genomic DNA
atggacagctacctgctgatgtggggcctgctgaccttcatcatggtgcctggctgtcaggcc

SEQ ID NO: 2 - protein, 21 aa, Homo sapiens
MDSYLLMWGLLTFIMVPGCQA

SEQ ID NO: 3 - DNA, 78 bp, Homo sapiens
  source 1..78: mol_type = genomic DNA
atggctgctccagctctgtcttggagactgcccctgctgattctgctgctgcctctggctacatcttgggcctctgcc

SEQ ID NO: 4 - protein, 26 aa, Homo sapiens
MAAPALSWRLPLLILLLPLATSWASA

SEQ ID NO: 5 - DNA, 66 bp, Homo sapiens
  source 1..66: mol_type = genomic DNA
atgctgaagcccagcctgccttttaccagcctgctgttcctgcagctgcctctgcttggcgtggga

SEQ ID NO: 6 - protein, 22 aa, Homo sapiens
MLKPSLPFTSLLFLQLPLLGVG

SEQ ID NO: 7 - DNA, 73 bp, Homo sapiens
  source 1..73: mol_type = genomic DNA
atggggtggctttgctctgggctcctgttccctgtgctgcctggtcctgctgcaggtggcaagctctgggaac

SEQ ID NO: 8 - protein, 25 aa, Homo sapiens
MGWLCSGLLFPVSCLVLLQVASSGN

SEQ ID NO: 9 - DNA, 60 bp, Homo sapiens
  source 1..60: mol_type = genomic DNA
atgacaatcctgggcaccaccttcggcatggtgttcagtctgctgcaggtcgtgtctggc

SEQ ID NO: 10 - protein, 20 aa, Homo sapiens
MTILGTTFGMVFSLLQVVSG

SEQ ID NO: 11 - DNA, 119 bp, Homo sapiens
  source 1..119: mol_type = genomic DNA
atgggactgggcagatgcatctgggaaggctggaccttggagagtgaggccctgaggcgagacatgggcactggctcctggcctgcatctgcatctgcacctgtgtctgcttgggagtc

SEQ ID NO: 12 - protein, 40 aa, Homo sapiens
MGLGRCIWEGWTLESEALRRDMGTWLLACICICTCVCLGV

SEQ ID NO: 13 - DNA, 90 bp, Homo sapiens
  source 1..90: mol_type = genomic DNA
atggctcctcggagagccagaggctgtagaacacttggactgcccgctctgctgctgctcctgcttcttagacctcctgccacaagaggc

SEQ ID NO: 14 - protein, 30 aa, Homo sapiens
MAPRRARGCRTLGLPALLLLLLLRPPATRG

SEQ ID NO: 15 - DNA, 57 bp, Homo sapiens
  source 1..57: mol_type = genomic DNA
atgccaagaggatgggccgctcctcttctcctgttgctgcttcaaggcggctggggc

SEQ ID NO: 16 - protein, 19 aa, Homo sapiens
MPRGWAAPLLLLLLQGGWG

SEQ ID NO: 17 - DNA, 57 bp, Homo sapiens
  source 1..57: mol_type = genomic DNA
gtggccgtggccggatgtgtgtttctgctgatctctgtgctgctgctgagcggcctg

SEQ ID NO: 18 - protein, 19 aa, Homo sapiens
VAVAGCVFLLISVLLLSGL

SEQ ID NO: 19 - DNA, 75 bp, Homo sapiens
  source 1..75: mol_type = genomic DNA
atcccctggctgggacatctgcttgttggactgtctggcgccttcggcttcatcatcctggtgtatctgctgatc

SEQ ID NO: 20 - protein, 25 aa, Homo sapiens
IPWLGHLLVGLSGAFGFIILVYLLI

SEQ ID NO: 21 - DNA, 63 bp, Homo sapiens
  source 1..63: mol_type = genomic DNA
gtggtcatctctgtgggctctatgggcctgatcatctccctgctgtgtgtgtacttttggctg

SEQ ID NO: 22 - protein, 21 aa, Homo sapiens
VVISVGSMGLIISLLCVYFWL

SEQ ID NO: 23 - DNA, 72 bp, Homo sapiens
  source 1..72: mol_type = genomic DNA
ctcctgctgggcgtcagcgtttcctgcattgtcatcctggccgtctgcctgttgtgctatgtcagcatcacc

SEQ ID NO: 24 - protein, 24 aa, Homo sapiens
LLLGVSVSCIVILAVCLLCYVSIT

SEQ ID NO: 25 - DNA, 75 bp, Homo sapiens
  source 1..75: mol_type = genomic DNA
cccatcctgctgacaatcagcatcctgagctttttcagcgtggccctgctggtcatcctggcctgtgtgctgtgg

SEQ ID NO: 26 - protein, 25 aa, Homo sapiens
PILLTISILSFFSVALLVILACVLW

SEQ ID NO: 27 - DNA, 63 bp, Homo sapiens
  source 1..63: mol_type = genomic DNA
ggcaacacccttgttgctgtgtccatctttctcctgctgactggcccgacctacctcctgttc

SEQ ID NO: 28 - protein, 21 aa, Homo sapiens
GNTLVAVSIFLLLTGPTYLLF

SEQ ID NO: 29 - DNA, 69 bp, Homo sapiens
  source 1..69: mol_type = genomic DNA
gtggccatcagcacaagcaccgttctgctgtgtggcctgtcagccgttagcctgctggcttgctacctg

SEQ ID NO: 30 - protein, 23 aa, Homo sapiens
VAISTSTVLLCGLSAVSLLACYL

SEQ ID NO: 31 - DNA, 63 bp, Homo sapiens
  source 1..63: mol_type = genomic DNA
ggctggaatcctcatctgttgttgttgctcctcctggtcatcgtgttcatccccgccttttgg

SEQ ID NO: 32 - protein, 21 aa, Homo sapiens
GWNPHLLLLLLLVIVFIPAFW

SEQ ID NO: 33 - DNA, 39 bp, Homo sapiens
  source 1..39: mol_type = genomic DNA
acttggcaaagacggcagagaaagagccggcggaccatc

SEQ ID NO: 34 - protein, 13 aa, Homo sapiens
TWQRRQRKSRRTI

SEQ ID NO: 35 - DNA, 858 bp, Homo sapiens
  source 1..858: mol_type = genomic DNA
aactgccggaacacaggcccttggctgaagaaagtgctgaagtgcaacacccctgatccgagcaagttctttagccagctgagcagcgagcatggcggcgacgttcagaaatggctgtctagcccatttcctagcagcagcttcagcccaggtggactggcccctgagattagccctctggaagtgctggaacgggacaaagtgacccagctgctcctccagcaggataaggtgccagaacctgccagcctgtccagcaatcacagcctgaccagctgctttaccaaccagggctacttcttcttccatctgcctgacgctctggaaatcgaggcctgccaggtgtacttcacctacgatccctacagcgaagaggaccccgatgaaggtgttgctggcgcccctacaggatcttctccacagcctctgcaacctctgagcggcgaggatgatgcctactgcacctttccaagcagggacgacctgctcctgttcagcccatctctgctcggaggaccatctcctccatctacagctccaggcggatctggcgctggcgaggaaagaatgccacctagcctgcaagagcgggtgcccagagattgggatcctcaacctctcggccctccaacacctggcgtgccagatctcgtggactttcagcctcctccagagctggtgctgagagaagctggcgaagaagtgccagacgctggccctagagagggcgttagctttccttggagcagacctcctggacagggcgagtttagggccctgaatgcaagactgcctctgaacaccgacgcctacctgtctctgcaagaactgcagggacaagaccccacacacctggtg

SEQ ID NO: 36 - protein, 286 aa, Homo sapiens
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 37 - DNA, 258 bp, Homo sapiens
  source 1..258: mol_type = genomic DNA
gaacggaccatgcctcggatccccacactgaagaacctggaagatctggtcaccgagtaccacggcaacttcagtgcttggagcggcgtgtcaaaaggactggccgaaagcctgcagcctgactactccgagagactgtgcctggtgtctgagatccctcctaaaggcggcgctctcggagaaggacctggtgcctctccatgcaatcagcacagcccttattgggcccctccttgctacaccctgaaacctgagaca

SEQ ID NO: 38 - protein, 86 aa, Homo sapiens
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 39 - DNA, 1707 bp, Homo sapiens
  source 1..1707: mol_type = genomic DNA
aagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctct

SEQ ID NO: 40 - protein, 569 aa, Homo sapiens
KIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 41 - DNA, 585 bp, Homo sapiens
  source 1..585: mol_type = genomic DNA
aagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggctttctgcaggacacattccctcagcagctggaagagagcgagaagcagagactcggaggcgacgtgcagagccctaattgcccttctgaggacgtcgtgatcacccctgagagcttcggcagagatagcagcctgacatgtctggccggcaatgtgtccgcctgtgatgcccctatcctgagcagcagcagaagcctggattgcagagagagcggcaagaacggccctcacgtgtaccaggatctgctcctgagcctgggaaccaccaatagcacactgcctccaccattcagcctgcagagcggcatcctgacactgaaccctgttgctcagggccagccaatcctgaccagcctgggcagcaatcaagaagaggcctacgtcaccatgagcagcttctaccagaaccag

SEQ ID NO: 42 - protein, 195 aa, Homo sapiens
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 43 - DNA, 690 bp, Homo sapiens
  source 1..690: mol_type = genomic DNA
aagctgtcacctagagtgaagcgcatattctatcagaatgtgcccagccctgcgatgttcttccagccattgtatagcgtacacaatggcaatttccagacctggatgggcgcccacggagctggtgttttgttgtctcaagattgtgcagggacgccccaaggcgcgttggagccttgtgtccaggaagcaaccgctcttctcacatgtggccctgctaggccatggaaaagcgtggctttggaggaggagcaagagggcccaggcacgagacttccaggaaacctctccagcgaggacgttctgcccgctggatgcacagaatggcgagtgcagacgctggcttatttgcctcaggaggactgggccccaacaagccttaccagacccgcgccaccagattcagagggaagccgatccagttctagctcctcatcaagcaacaataacaattattgcgctctgggatgttatggtggatggcacttgagcgcgttgccaggcaacacgcaatcatctggtcccatacccgcgcttgcatgcggactgagttgcgaccatcagggtctcgaaactcagcaaggtgttgcgtgggtcctggcgggtcattgccaaagacctggcctgcacgaggatcttcagggaatgcttttgccaagtgtgctctccaaggctcgatcttggacgttc

SEQ ID NO: 44 - protein, 230 aa, Homo sapiens
KLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 45 - DNA, 123 bp, Homo sapiens
  source 1..123: mol_type = genomic DNA
aagtccagacagacacctcctctggccagcgtggaaatggaagccatggaagctctgccagtgacctggggcacctccagcagagatgaggatctggaaaactgcagccaccacctgtgatga

SEQ ID NO: 46 - protein, 39 aa, Homo sapiens
KSRQTPPLASVEMEAMEALPVTWGTSSRDEDLENCSHHL

SEQ ID NO: 47 - DNA, 854 bp, Homo sapiens
  source 1..854: mol_type = genomic DNA
agcctgaaaacacaccctctgtggcggctgtggaagaaaatctgggccgtgccatctcctgagcggttcttcatgcctctgtacaagggctgcagcggcgacttcaagaaatgggtcggagccccttttaccggcagctctctggaacttggaccttggagccctgaagtgcccagcacactggaagtgtacagctgtcaccctcctagaagccccgccaagagactgcagctcacagagctgcaagagcctgccgagctggtggaatctgatggcgtgcccaagcctagcttctggcccaccgctcaaaattctggcggcagcgcctacagcgaggaaagagatagaccttacggcctggtgtccatcgacaccgtgacagtgctggatgccgagggaccttgtacctggccttgtagctgcgaggacgatggctaccctgctctggatctggacgcaggactggaaccttctccaggcctcgaagatcctctgctggacgccggaacaacagtgctgtcttgtggctgtgtgtccgccggatctcctggacttggaggacctctgggaagcctgctggacagactgaaacctcctctggccgatggcgaagattgggctggtggacttccttggggcggaagatctccaggcggagtgtctgaatctgaggccggttctccactggccggcctggatatggataccttcgatagcggcttcgtgggcagcgattgcagcagccctgtggaatgcgacttcacatctcctggcgacgagggcccacctagaagctatctcagacagtggtcgtgatccctccacctctgtctagtcctggaccacaggccagc

SEQ ID NO: 48 - protein, 285 aa, Homo sapiens
SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 49 - DNA, 135 bp, Homo sapiens
  source 1..135: mol_type = genomic DNA
acaacaacccctgctcctcggcctcctacaccagctcctacaattgccagccagccactgtctctgaggcccgaagcttgtagacctgctgcaggcggagccgtgcatacaagaggactggacttcgcctgtgac

SEQ ID NO: 50 - protein, 45 aa, Homo sapiens
TTTPAPRPPTPAPTIASQPLSLRPEACRPAAGGAVHTRGLDFACD

SEQ ID NO: 51 - DNA, 762 bp, Homo sapiens
  source 1..762: mol_type = genomic DNA
gacgtggtcatgacacagacccctctgtctctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtct

SEQ ID NO: 52 - protein, 254 aa, Homo sapiens
DVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVS

SEQ ID NO: 53 - DNA, 729 bp, Homo sapiens
  source 1..729: mol_type = genomic DNA
caagtgcggctgcaagagtctggacctagcctggtcaagcccagccagacactgagcctgacctgtaccgtgtccggcttcagcctgaccaactactatgtcggctgggtccgacaggcccctggaaaagctcttgagtgggtcggagtgatggatagcggcggaggcacctactacaaccccgctctgaaaagccggctgagcatcaccagagacaccagcaagtctcaggtgtccctgagcctgtctagcgtgaccacagaggataccgccgtgtactactgcgccagcttcagcttcggcagagactggaattattggggccctggcctgctgctgacaatcccttctgagggcaagtctagcggcagcggctctgagagcaaggtggacagctatgagctgacccagcctagcagcgtgtccagatctctgggccagtccgtgtccatcacctgtagcggaagcagcagcaatgtcggctacggcaattacgtcggctggttccagcaggtcccaggctctgctcctaagctgctgatctacgacgccacctctagagccagcggcgtgccagatagattcagcggcagcagaagcagcaacaccgccacactgacaatcagcagtctgcaggccgaggacgaggccgattactattgtgcccactgggacagcagcgccaatatcgccatctttggctccggcagcaggctgacagttctggga

SEQ ID NO: 54 - protein, 243 aa, Homo sapiens
QVRLQESGPSLVKPSQTLSLTCTVSGFSLTNYYVGWVRQAPGKALEWVGVMDSGGGTYYNPALKSRLSITRDTSKSQVSLSLSSVTTEDTAVYYCASFSFGRDWNYWGPGLLLTIPSEGKSSGSGSESKVDSYELTQPSSVSRSLGQSVSITCSGSSSNVGYGNYVGWFQQVPGSAPKLLIYDATSRASGVPDRFSGSRSSNTATLTISSLQAEDEADYYCAHWDSSANIAIFGSGSRLTVLG

SEQ ID NO: 55 - DNA, 747 bp, Homo sapiens
  source 1..747: mol_type = genomic DNA
caagttcggctgcaaggctctggacctagcctggtcaagcctagccagacactgagcctgacctgtaccgtgtccggcttcagcctgacaagcaacgctgtggactgggtccgacaggctcctggaaaagtgcctgagtggctgggcttcatcagaggcggcggaagcaccttctacaacagcgccctgaagtccagactgagcatcaccagagacaccagcaagagccaggtgtccctgagcctgtctagcgtgaccacagaggacaccgccgtgtactactgtgccagagccagctgtagcggcgacatctacacagacacctgtggcatcgactactggggccctggactgctggttaccgttagctctgagggcaagtctagcggcagcggaagcgagagcaaagtggaccagtctgccctgacacagcctagcagcgtgtccagatctctgggccagtccgtgtccatcacctgttctggcagcagctctaacgtcggcgctggcaactacgtgaactggttcagactgatccctggcagcgcccctaagagcctgatctacgctgccacaacaagagcctctggcgtgcccgatagattcagcggctctagaagcggcaacaccgccacactgacaatcagcagtctgcaggccgaggacgaggccgactactactgtagcagctacgacatcacagccgtgaacctgttcggctccggcaccagactgacagttcttgga

SEQ ID NO: 56 - protein, 249 aa, Homo sapiens
QVRLQGSGPSLVKPSQTLSLTCTVSGFSLTSNAVDWVRQAPGKVPEWLGFIRGGGSTFYNSALKSRLSITRDTSKSQVSLSLSSVTTEDTAVYYCARASCSGDIYTDTCGIDYWGPGLLVTVSSEGKSSGSGSESKVDQSALTQPSSVSRSLGQSVSITCSGSSSNVGAGNYVNWFRLIPGSAPKSLIYAATTRASGVPDRFSGSRSGNTATLTISSLQAEDEADYYCSSYDITAVNLFGSGTRLTVLG

SEQ ID NO: 57 - DNA, 774 bp, Homo sapiens
  source 1..774: mol_type = genomic DNA
gcctctcacgtgaagctgcaagagtctggacctggcctggtgcagcctagccaaagcctgtctctgacctgtaccgtgtccggcttcagcctgacagattacggcgtgcactgggtccgacagagccctggaaaaggactggaatggctgggagtgatttggagcggcggaggcacagcctataacacagccctgatcagcagactgaacatctaccgggacaacagcaagaaccaggtgttcctggaaatgaactccctgcaggccgaggacaccgccatgtactactgtgccagaagaggcagctacccctacaactacttcgacgcctggggctgtggcaccaccgtgacagtttctagcggaggcggaggatctggtggcggaggtagtggtggcggtggatctcaggccgtggtcatccaagaaagcgccctgacaacacctcctggcgagacagtgacactgacctgtggatcttctacaggcgccgtgaccgccagcaactacgccaattgggtgcaagagaagcccgaccactgcttcacaggcctgatcggcggccacaacaatagacctccaggcgtgccagccagattcagcggatctctgatcggagacaaggccgctctgacaatcgccggcacacagacagaggacgaggccatctacttttgcgccctgtggtacagcgaccactgggttatcggcggaggaaccagactgacagtgctcggcggatctgagcagaagctgatctccgaagaggacctg

SEQ ID NO: 58 - protein, 258 aa, Homo sapiens
ASHVKLQESGPGLVQPSQSLSLTCTVSGFSLTDYGVHWVRQSPGKGLEWLGVIWSGGGTAYNTALISRLNIYRDNSKNQVFLEMNSLQAEDTAMYYCARRGSYPYNYFDAWGCGTTVTVSSGGGGSGGGGSGGGGSQAVVIQESALTTPPGETVTLTCGSSTGAVTASNYANWVQEKPDHCFTGLIGGHNNRPPGVPARFSGSLIGDKAALTIAGTQTEDEAIYFCALWYSDHWVIGGGTRLTVLGGSEQKLISEEDL

SEQ ID NO: 59 - protein, 352 aa, synthetic construct
MDSYLLMWGLLTFIMVPGCQADVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSTTTPAPRPPTPAPTIASQPLSLRPEACRPAAGGAVHTRGLDFACDVAVAGCVFLLISVLLLSGLTWQRRQRKSRRTI

SEQ ID NO: 60 - DNA, 1635 bp, synthetic construct
  source 1..1635: mol_type = other DNA
atggctctgcctgtgacagctctgctgctgcctctggctctgcttctgcatgccgccagacctgacgtggtcatgacacagacacctctgagcctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggatttcgcctgcgacttctgggtgctcgtggttgttggcggagtgctggcttgttactccctgctggttaccgtggccttcatcatcttttgggtccgaagcaagcggagccggctgctgcacagcgactacatgaacatgacccctagacggcccggacctaccagaaagcactaccagccttacgctcctcctagagacttcgccgcctacagatccaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgagatgggcggaaagccccagcggagaaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgccacctagatgatga

SEQ ID NO: 61 - DNA, 1641 bp, synthetic construct
  source 1..1641: mol_type = other DNA
atgggtttcactacgaaaattatctttctgtataatctggtactcgtatatgcgggtttcgacgatcccaggaaagcgatcgaacttgtccagaagagatacgggaggccctgtgactgcagcggagggcaagtatcagaacccccctctgatcgggtcagccaagttacttgcagcggcaaaacagcttacctgatgccggatcagagatggaaatgcaaatccatacccaaggacaccagtccgagtggaccattgcaggaatgtccgtgtaatagttaccaatcaagcgtccattcaagttgctacacgtcataccagcaatgtcgctcaggaaataaaacctattatacggcgacactgcttaaaacccaaacgggtggcacctctgatgttcaggttctcggaagtacgaataagttgattcagagtccctgcaacggtatcaaaggccagtcaatttgttggtctacgacagcgcctatccatgtgagtgacggcggtgggccgttggatacaacacgaataaaaagtgtacagcggaaacttgaggagatacacaaagccctctaccccgagcttcagtaccatcccctggccatccctaaggtcagggacaatctcatggtagacgctcaaaccctcaacatcctcaatgccacctacaatctcttgttgatgtctaacacaagcttggtagatgactgctggctctgtcttaaattgggccctccgactcccctcgctatacccaacttccttctgtcatacgtaacgcgcagctccgacaacatatcatgtctgataatcccgccgttgcttgtgcagcccatgcagttctctaacagctcctgcttgttcagtccatcttataattcaacagaagaaattgatttgggccatgtagctttcagtaactgtacatcaataactaacgtcactggccccatctgcgccgtgaacggttctgtcttcctctgcggcaacaatatggcttatacatacttgccaactaactggaccggtctgtgtgtattggccacgctgttgcctgacatagatataatccctggcgacgaacccgtccctatcccagccatcgaccattttatttatcgccccaagcgcgcgattcagtttatccctctgctcgctgggttgggcattacggctgcttttactacgggggctaccggccttggagtgtccgttacccaatatacgaaactgtccaatcaattgatttcagacgtgcaaatcttgagctctactatccaggatctgcaggaccaggtagactctctggcggaagtcgtcttgcaaaatcggcgggggttggatctgctgaccgccgagcagggcggcatctgtcttgctcttcaagaaaaatgctgtttttacgtgaacaaatcaggtattgtaagagataaaataaaaactttgcaagaagagctcgaaaggaggcggaaagacctggcgtctaatcctctgtggactggcctgcaggggctcctcccctatttgctgccctttcttggtccgctcctgactttgttgctgctcctgactattgggccatgcatcttcaatcgactcaccgcgttcatcaatgataaactcaacataatccacgctatgtga

SEQ ID NO: 62 - protein, 546 aa, synthetic construct
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAM

(19) United States
(12) Patent Application Publication
(10) Pub. No.: US 2024/0408139 A1
(43) Pub. Date: Dec. 12, 2024

(54) ENGINEERED CYTOKINE RECEPTORS FOR TUNABLE ADOPTIVE CELL THERAPY

(71) Applicant: Dana-Farber Cancer Institute, Inc., Boston, MA (US)

(72) Inventors: Carl NOVINA, Newton, MA (US); Aya KOBAYASHI, Brookline, MA (US); Robert J. DISTEL, Framingham, MA (US); Alberto NOBILI, Brookline, MA (US); Steven NEIER, Waltham, MA (US)

(21) Appl. No.: 18/699,108

(22) PCT Filed: Oct. 5, 2022
(86) PCT No.: PCT/US2022/077610

§ 371 Date: Apr. 5, 2024

Related U.S. Application Data
(60) Provisional application No. 63/252,850, filed on Oct. 6, 2021.

(51) Int. Cl. A61K 35/17; A61K 39/00; C07K 14/715; C07K 16/44

(52) U.S. Cl. CPC A61K 35/17; A61K 39/4611; A61K 39/4613; A61K 39/4631; A61K 39/4637; C07K 14/7155; C07K 16/44; C07K 2317/53; C07K 2317/622; C07K 2319/02; C07K 2319/03

(57) ABSTRACT

Disclosed are receptor cytokine switches, immune cells containing them, and uses thereof for controllable adoptive cell therapy.

### RELATED APPLICATIONS

This application claims the benefit of priority under 35 U.S.C. § 119 (e) to U.S. Provisional Application No: 63/252,850, filed Oct. 6, 2021, which is incorporated herein by reference in its entirety.

### GOVERNMENT LICENSE RIGHTS

This disclosure was made with government support under grant number 1DP1DK105602-01 awarded by the National Institutes of Health. The government has certain rights in the disclosure.

### SEQUENCE LISTING

The instant application contains a Sequence Listing which has been submitted electronically in XML format and is hereby incorporated by reference in its entirety. Said XML copy, created on Aug. 27, 2022, is named 52095-631001WO_ST.xml and is 69 KB bytes in size.

### BACKGROUND OF THE DISCLOSURE

Immunotherapy using adoptive cell transfer (ACT) aims to stimulate or suppress immunity. One of the most promising approaches of ACT is the administration of antigen-specific T or chimeric antigen receptor-T (CAR T) cells. CAR T cells targeting the B cell antigen CD19 have had remarkable clinical responses in patients with B cell malignancies (Davila and Brentjens, Clin. Adv. Hematol. Oncol. 14 (10): 802-808 (2016): Halim and Maher, Ther. Adv. Vaccines Immunother. 8:1-17 (2020)). Despite successes in hematological cancers, the effect of CAR-T cells against solid tumors has been limited partly because of the limited persistence, survival, expansion, and the efficacy of CAR-T cells after infusion (Kosti, et al., Front. Immunol. 9:1104-9 (2018): Jafarzadeh, et al., Front. Immunol. 11:702-17 (2020).

Generation of memory T cells is essential for prolonged persistence of CAR T cell therapy. Their formation requires three signals, namely antigen, co-stimulation and pro-inflammatory cytokines. Cytokines have crucial roles in the development, proliferation, survival, and differentiation of various immune cells (Foster, Int. J. Exp. Pathol. 82 (3): 171-192 (2001)). Cytokine-targeted immunotherapies can modulate immune responses by promoting or inhibiting specific immune-cell functions. Interleukin-2 (IL-2) stimulates T cell development and survival. High-dose IL-2 therapy was approved by the United States Food and Drug Administration (FDA) for treatment of metastatic melanoma and kidney cancer (Jiang, et al., Oncoimmunology 5 (6): e1163462-10 (2016)). However, systemic administration of cytokines has been largely unsuccessful due to uncontrolled cytokine release syndrome and intolerable toxicity. Therefore, controllable activation of cytokine signaling in specific cells is necessary to prevent systemic toxicity and to improve engineered immune cell-driven efficacy.

### SUMMARY OF THE DISCLOSURE

A first aspect of the present disclosure is directed to a cytokine receptor switch comprising a signal peptide, a single chain antibody fragment (scFv) that specifically binds a synthetic, substantially nonimmunogenic small molecule (hereinafter “synthetic small molecule”), a hinge domain, a transmembrane domain, and an intracellular domain of a cytokine receptor.

In some embodiments, the signal peptide is native to or derived from a cytokine receptor in which case the signal peptide and the intracellular domain may be native to or derived from different cytokine receptors. In some embodiments, the signal peptide is native to or derived from interleukin-2 receptor alpha chain (IL-2RA), IL-2RB, IL-2RG, IL-4RA, IL-7RA, IL-9R, IL-15RA, or IL-21R. In other embodiments, the signal peptide is native to or derived from cluster of differentiation 8 (CD8).

In some embodiments, the synthetic small molecule that specifically binds the scFv is a fluorescein (e.g., fluorescein isothiocyanate (FITC)), 4-[(6-methylpyrazin-2-yl) oxy] benzoate (MPOB), anthraquinone-2-carboxylate (AQ), tetraxetan (DOTA), or polyhistidine-tag (His-tag).

In some embodiments, the hinge domain is native to or derived from CD8.

In some embodiments, the transmembrane domain and intracellular domain are each independently native to or derived from IL-2RA, IL-2RB, IL-2RG, IL-4RA, IL-7RA, IL-9R, IL-15RA, or IL-21R.

Another aspect of the present disclosure is directed to nucleic acids encoding the cytokine receptor switches described herein.

A further aspect is directed to a composition comprising an immune cell that comprises an exogenous nucleic acid encoding the cytokine receptor switch. In some embodiments, the immune cells are T cells such as CD8⁺ and CD4⁺ T cells. In other embodiments, the immune cells are NK cells.

In some embodiments, the immune cell comprises at least two nucleic acid which encode that encode different cytokine receptor switches that comprise different signal peptides from different cytokine receptors, different transmembrane domains, and/or different intracellular domains of the cytokine receptors.

In some embodiments, the immune cells contain an exogenous nucleic acid that encodes a chimeric antigen receptor (CAR) directed against a cell surface antigen. In some embodiments, the cell surface antigen is CD19, B-cell maturation antigen (BCMA), human epidermal growth factor receptor 2 (HER2), epidermal growth factor receptor (EGFR), mucin 1 (MUC1), or TNF receptor superfamily member 13B (TNFRSF13B).

In some embodiments, the immune cells comprise a nucleic acid that encodes a binary activated chimeric antigen receptor (BAT-CAR).

In some aspects, the present disclosure is directed to a method for stimulating an immune cell that comprise an exogenous nucleic acid that encodes the cytokine receptor switch, comprising contacting the immune cells with a sufficient concentration of the synthetic, small molecule, wherein the contacting promotes proliferation of the immune cells. It may also promote a change in phenotype (e.g., memory, cytotoxic, and regulatory phenotypes) of the immune cells.

In some embodiments, the contacting is conducted ex vivo. Immune cells that contain an exogenous nucleic acid encoding the cytokine receptor switch are placed in suitable medium and contacted with an effective concentration of the synthetic small molecule, e.g., from 0.1 to 1000 μg/mL based on total volume of the medium. In some embodiments, the concentration of the synthetic small molecule ranges from 0.1 to 100 μg/mL based on total volume of the medium. The duration of the contact (also referred to herein as “treatment” or “treating”) may be in the order of hours, days and weeks (e.g., 1, 2, 3, 4 or more weeks). In some embodiments, the contacting may be conducted in a high-affinity plate, dish, or flask wherein the synthetic small molecule is conjugated to the carrier, which may be polymeric in nature, e.g., bovine serum albumin (BSA) affixed to a surface thereof.

In some other embodiments, the immune cells are contacted with the synthetic small molecule in vivo. In some embodiments, the immune cells are contacted with the synthetic small molecule systemically. In other embodiments, the immune cells are CAR-containing immune cells that due to the binding specificity of the CAR, are directed to specific cell surface antigens, e.g., tumor surface antigens, thus allowing for local stimulation of the immune cells. Further aspects of the present disclosure are directed to methods of treating cancer. In some embodiments, a therapeutically effective concentration of a composition containing the ex-vivo-stimulated immune cells are administered to a subject. In other embodiments, the method comprises: administering to a subject in need thereof a therapeutically effective concentration of immune cells: administering to the subject an effective concentration of a synthetic small molecule for a suitable time period, thereby stimulating the immune cells: and decreasing the stimulation of the immune cells by administering to the subject a composition containing synthetic monomeric or polymeric small molecules. To optimize stimulation, the synthetic small molecules are advantageously administered as multimers, e.g., wherein monomers of the small molecule are conjugated to a carrier such as BSA.

In some embodiments, the cancer is a hematological cancer such as leukemia, lymphoma or multiple myeloma. In some embodiments, the cancer is characterized by the presence of a solid tumor such as breast cancer, ovarian cancer, lung cancer, or brain cancer, e.g., glioblastoma multiforme.

Methods of stimulating immune cells comprising nucleic acids encoding cytokine receptor switches are known in the art, as described for example, in U.S. Pat. No. 5,747,292 and International Publication Nos. WO/2018/111834 and WO 2019/193197, each of which is incorporated herein by reference in its entirety. Methods are making and stimulating immune cells comprising nucleic acids encoding cytokine receptor switches are also described in Nelson et al., Nature, 369 (6478): 333-336 (1994): Gerhartz et al., J. Biol. Chem., 277: 12991-12998(1996): Sockolosky et al., Science, 2018, 359 (6379): 1037-1042 (1996): Chang et al., Nat. Chem. Biol. 14 (3): 317-324 (2018), Leung et al., JCI Insight 4 (11): e124430-18 (2019), and Yang et al., PNAS 118: e2106612118-12 (2021).

The inventive cytokine receptor switches differ from known chimeric cytokine receptors. For example, chimeric receptors taught in U.S. Pat. No. 5,747,292 are activated by endogenous proteins which cross-react with natural receptors. The chimeric receptors taught in WO 2019/193197 are activated by bacterial proteins (e.g., GFP and mCherry) that are not substantially nonimmunogenic (i.e., the bacterial proteins GFP and mCherry are immunogenic).

The present disclosure provides compositions and methods to direct controllable cytokine receptor signaling to a desired set of immune cells independent of their natural ligands. In contrast with the prior art, the present disclosure requires use of a synthetic small molecule that serves as an artificial ligand and a substitute for the native cytokine, and which is also substantially nonimmunogenic. Therefore, the cytokine receptor switch is under the control of an exogenous moiety, namely the synthetic small molecule, allowing for precise control of activation or stimulation of the cytokine receptor and the ensuing signaling, both ex vivo and in vivo. Basically, the synthetic small molecule acts as an on/off switch.

The present disclosure may offer several additional advantages, especially in the context of CAR-T cells. Combinations of different cytokine receptor switches and CARs may pre-condition T cells with memory, effector, or regulatory phenotypes, which may greatly improve persistency and cytotoxicity of the CAR T cells. In addition to controlling their activation or stimulation, the CAR-T cells that contain a cytokine receptor switch may exhibit greater resistance to the immunosuppressive environments characteristic of solid tumor microenvironments. As shown in working examples below, T cell subsets expressing memory markers were increased using compositions of the present disclosure. Use of synthetic small molecules provide additional advantages, particularly with respect to ex vivo expansion of CAR-T cells that contain the cytokine receptor switch, namely in terms of cost relative to use of antibodies and recombinant cytokines.

### BRIEF DESCRIPTION OF THE DRAWINGS

FIGS. 1A-1D are images showing structures and primary sequences for inventive cytokine receptor switches. FIG. 1A is an image showing the components of a cytokine receptor switch. A synthetic small molecule serves as an artificial ligand instead of a natural cytokine of the cytokine receptor. FIG. 1B is an image showing systemic and local activations of cytokine receptor switches with small molecules conjugated to nonimmunogenic scaffold or antibodies. Cytokine receptor switches may be combined with a chimeric antigen receptor (CAR). Either the same single chain antibody against a synthetic small molecule (scFv) against the same small molecule (homotargeted) or different scFvs against different small molecules (heterotargeted) were fused in the cytokine receptor switches or CAR. FIG. 1C is an image showing a representative primary sequence for the inventive cytokine receptor switches. FIG. 1D is an image showing primary sequences for inventive cytokine receptor switches.

FIG. 2 is an image showing cytokine receptor switches expressed on primary human T cells that were stimulated by fluorescein isothiocyanate (FITC) conjugated to bovine serum albumin (BSA) coated on a culture plate.

FIGS. 3A-3D are sets of graphs showing that inventive cytokine receptor switches increased effector and central memory markers on T cells with CD3/CD28-costimulation. FIG. 3A is a set of graphs showing that inventive IL2RA-and IL15RA-cytokine receptor switches increased effector memory markers on CD4⁺ T cells with CD3/CD28-costimulation, and IL2RA-, IL2RB-, IL2RG-, IL7RA-, and IL15RA-cytokine receptor switches increased effector memory markers on CD8⁺ T cells with CD3/CD28-costimulation. FIG. 3B a set of graphs showing that inventive IL2RA-and IL7RA-cytokine receptor switches increased central memory markers on CD8⁺ T cells with CD3/CD28-costimulation. FIG. 3C is a set of graphs that show that inventive cytokine receptor switches had little effect on effector memory markers CD4⁺ and CD8⁺ T cells without CD3/CD28-costimulation. FIG. 3D is a set of graphs showing that inventive IL7RA-cytokine receptor switch increased central memory markers on CD4⁺ T cells without CD3/CD28-costimulation.

FIG. 4 is an image showing the use of inventive cytokine receptor switches with CARs or binary activated T cells comprising nucleic acids encoding chimeric antigen receptors (BAT-CARs).

FIGS. 5A-5C are a set of graphs showing that BSA-FITC bound on high-affinity plate efficiently stimulated fluorescein-specific BAT-CAR-T cells. FIG. 5A is graph showing the expression of interleukin-2 in BAT-CAR-CD8⁺ T cells stimulated by BSA-FITC bound on normal tissue culture plate (plate-bound (normal)), BSA-FITC bound on high-affinity plate (plate-bound high affinity), and BSA-FITC solution added to directly cells (free). FIG. 5B is graph showing the expression of interferon gamma (IFNg)⁺ in BAT-CAR-CD8⁺ T cells stimulated by BSA-FITC bound on normal tissue culture plate, BSA-FITC bound on high-affinity plate, and BSA-FITC solution added to directly cells. FIG. 5C is graph showing the expression of CD69 on BAT-CAR-CD8⁺ T cells stimulated by BSA-FITC bound on normal tissue culture plate, BSA-FITC bound on high-affinity plate, and BSA-FITC solution added directly to the cells.

FIGS. 6A-6B are a set of graphs showing that inventive cytokine receptor switches activated NK cells. FIG. 6A is a line graph showing that the combination of inventive IL15RA-, IL2RB-and IL2RG-cytokine receptor switches promoted cell proliferation of human natural killer (NK) cells, NK92. FIG. 6B is a bar graph showing that the combination of inventive IL7RA-and IL2RG-cytokine receptor switches increased the expression of activation marker CD69 on NK92 cells.

### DETAILED DESCRIPTION OF THE DISCLOSURE

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present disclosure.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed disclosure.

As used herein, “immune cell” refers to a cell of hematopoietic origin functionally involved in the initiation and/or execution of innate and/or adaptative immune response. Representative examples of immune cells include T cells and natural killer (NK) cells. The term “immune cell” as used herein also refers to cells derived from stem cells. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells.

In some embodiments, the immune cells are CD8⁺ T cells. In some embodiments, the immune cells are CD4⁺ T cells. In some embodiments, the immune cells are a combination of CD8⁺ T cells and CD4⁺ T cells. In some embodiments, the immune cells are NK cells.

The term “memory phenotype”, as used herein, refers to a readiness of the immune cell that comprises an exogenous nucleic acid encoding an inventive cytokine receptor switch to respond to an antigen more quickly than a naïve immune cell (that does not contain the exogenous nucleic acid).

The term “cytotoxic phenotype” as used herein refers to immune cells that are toxic, i.e., immune cells that induce the death of other cells such as tumor cells, infected cells or cells that are otherwise damaged or dysfunctional. For example, cytotoxic T cells mediate the lysis of target cells (e.g., cancer cells) bearing cognate antigens. Cytotoxic T cells are generally antigen-specific and major histocompatibility complex (MHC)-restricted in that they recognize antigenic peptides only in association with the MHC molecules on the surface of target cells. NK cells target and kill aberrant cells including stressed, virus-or microbe-infected cells or malignant cells.

The term “antibody” is used herein in the broadest sense and includes polyclonal and monoclonal antibodies, including intact antibodies and functional (antigen-binding) antibody fragments, including fragment antigen binding (Fab) fragments, F (ab′)₂ fragments, Fab′ fragments, Fv fragments, recombinant IgG (rIgG) fragments, variable heavy chain (VH) regions capable of specifically binding the antigen, single chain antibody fragments, including single chain variable fragments (scFv), and single domain antibodies (e.g., sdAb, sdFv, nanobody) fragments. The scFv includes the variable regions of the VH and light chains (VL) of an antibody, and typically includes up to about 50, e.g., about 10 amino acid residues. The linker can either connect the N-terminus of the VH with the C-terminus of the VL, or vice versa. ScFvs can be prepared according to methods known in the art (see, e.g., Bird et al., Science 242:423-426 (1988) and Huston et al., Proc. Natl. Acad. Sci. USA 85:5879-5883 (1988)). In some embodiments, the linker sequence includes amino acids glycine and serine, and in some cases, sets of glycine and serine repeats such as (Gly4Ser)n, where n is an integer equal to or greater than 1. The length and amino acid composition of the linker may be varied e.g., to achieve optimal folding and interaction between the VH and VL to create a functional epitope. See, e.g., Hollinger et al., Proc. Natl. Acad. Sci. U.S.A. 90:6444-6448 (1993). The term “antibody” encompasses genetically engineered and/or otherwise modified forms of immunoglobulins, such as intrabodies, peptibodies, chimeric antibodies, fully human antibodies, humanized antibodies, and heteroconjugated antibodies, multispecific, e.g., bispecific, antibodies, nanobodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

As used herein, “synthetic substantially nonimmunogenic small molecule” refers to an organic molecule or compound that is monofuctional and that ranges in size from about 50 to about 10,000 daltons, usually from about 50 to about 5,000 daltons and more usually from about 100 to about 1000 daltons that can bind an scFy on the inventive cytokine receptor switches without provoking a significant immune response in the subject.

Examples of substantially nonimmunogenic small molecules as used herein include fluorescein and fluorescein derivatives (e.g., fluorescein isothiocyanate (FITC)), 4-[(6-methylpyrazin-2-yl) oxy] benzoate (MPOB), anthraquinone-2-carboxylate (AQ), tetraxetan (DOTA), or polyhistidine-tag (His-tag). A “significant immune” response is any immune response that would limit or restrict the in vivo utility of the synthetic small molecule as used in accordance with the teachings of the present disclosure. A detectable immune response is not necessarily a “significant immune response.” That is, “substantially nonimmunogenic” embraces immune responses that are detectable but not significant. In some embodiments, the substantially nonimmunogenic small molecule is conjugated to a carrier.

With respect to synthetic small molecules described herein, and to the extent the following terms are used herein to further describe them, the following definitions apply.

The term “specific binding” as it relates to interaction between the synthetic small molecule and the single chain antibody fragment (scFv) refers to an inter-molecular interaction that is substantially specific in that binding of the synthetic small molecule with other endogenous entities (proteinaceous and non-proteinaceous alike), although detectable, may be functionally insignificant.

As used herein in the context of elements of the cytokine receptor switch, CARs and BAT-CARs, the term “derived from” (also referred to as “derived from, e.g., native to”) embraces an element having a at least a portion of a sequence identical to the sequence of that element in a native gene, e.g., a signal peptide natively associated with an IL-2RA receptor (e.g., a signal peptide “native to” an IL-2RA receptor), and elements that have non-naturally occurring sequences that differ from a native sequence in terms of at least one modification such as an amino acid substitution, addition (e.g., at either or both termini) or deletion (e.g., “derived from”), provided that the modification does not compromise the function that the element performs as part of the cytokine receptor switch. In some embodiments, an element of a cytokine receptor switch, CAR, and BAT-CAR derived from a protein includes a sequence a portion of a native gene that has one or more alterations from the native gene or may be identical to the native gene for a range of amino acids or nucleic acids but has alterations, substitions, or deleteions outside of the range of identical sequence.

A used herein the term “sufficient concentration” refers to the concentration of a synthetic small molecule needed to activate a cytokine switch in the context in which the synthetic small molecule is being used (e.g., in vitro, or in vivo).

### Cytokine Receptor Switches

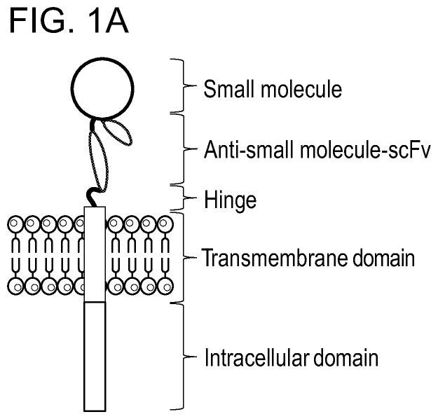

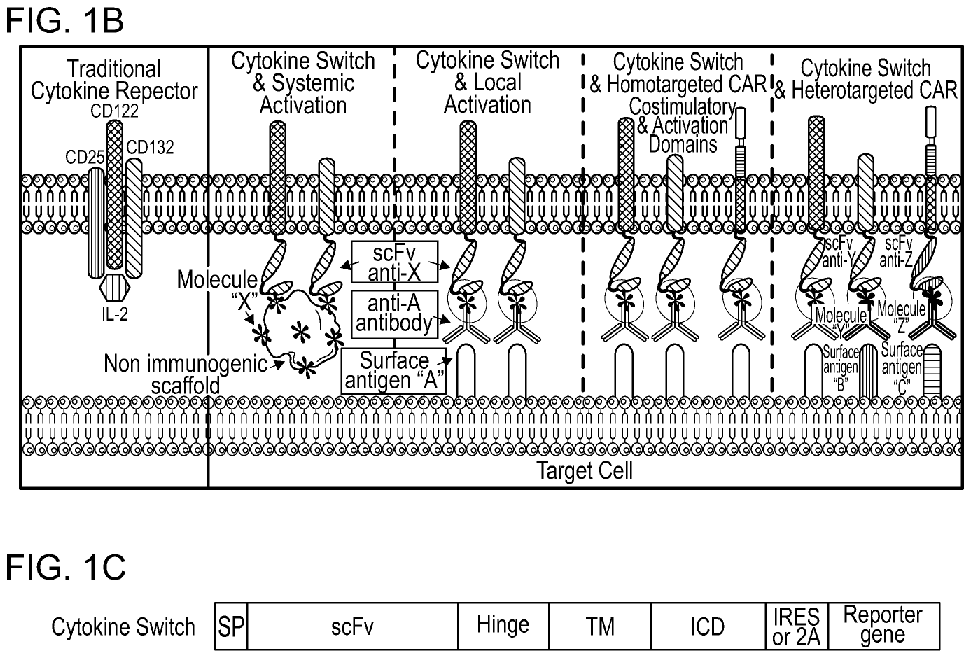

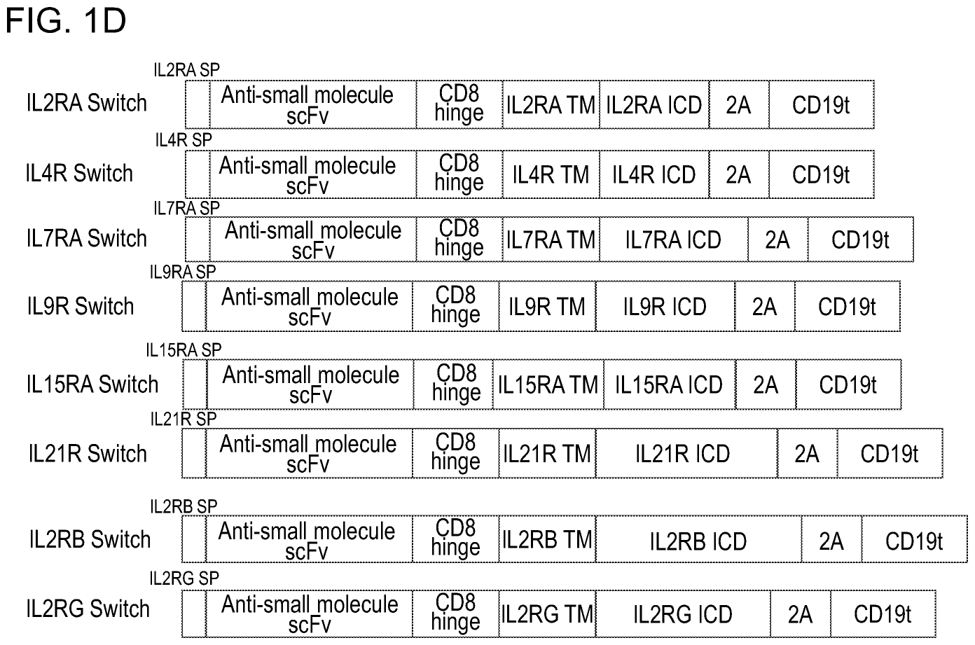

A key aspect of the present disclosure is directed to cytokine receptor switch comprising a signal peptide, e.g., which is native to or derived from cytokine receptor, a single chain antibody fragment (scFv) that specifically binds a synthetic small molecule, a hinge domain, a transmembrane domain, and an intracellular domain of a cytokine receptor (FIG. 1A, FIG. 1C, and FIG. 1D). DNA and amino acid sequences of representative components of cytokine receptor switches described herein are set forth in Table 1.

TABLE 1

| Domain | DNA Sequence | Amino Acid Sequence |
|---|---|---|
| IL2RA signal | ATGGACAGCTACCTGCTGATGTGG | MDSYLLMWGLLTFIMV |
| peptide | GGCCTGCTGACCTTCATCATGGTG | PGCQA (SEQ ID NO: 2) |
| | CCTGGCTGTCAGGCC | |
| | (SEQ ID NO: 1) | |
| IL2RB signal | ATGGCTGCTCCAGCTCTGTCTTGGA | MAAPALSWRLPLLILLL |
| peptide | GACTGCCCCTGCTGATTCTGCTGCT | PLATSWASA |
| | GCCTCTGGCTACATCTTGGGCCTCT | (SEQ ID NO: 4) |
| | GCC (SEQ ID NO: 3) | |
| IL2RG signal | ATGCTGAAGCCCAGCCTGCCTTTT | MLKPSLPFTSLLFLQLPL |
| peptide | ACCAGCCTGCTGTTCCTGCAGCTG | LGVG (SEQ ID NO: 6) |
| | CCTCTGCT TGGCGTGGGA | |
| | (SEQ ID NO: 5) | |
| IL4RA signal | ATGGGGTGGCTTTGCTCTGGGCTC | MGWLCSGLLFPVSCLV |
| peptide | CTGTTCCCTGTGCTGCCTGGTCCTG | LLQVASSGN |
| | CTGCAGGTGGCAAGCTCTGGGAAC | (SEQ ID NO: 8) |
| | (SEQ ID NO: 7) | |
| IL7RA signal | ATGACAATCCTGGGCACCACCTTC | MTILGTTFGMVFSLLQV |
| peptide | GGCATGGTGTTCAGTCTGCTGCAG | VSG (SEQ ID NO: 10) |
| | GTCGTG TCTGGC (SEQ ID NO: 9) | |
| IL9R signal | ATGGGACTGGGCAGATGCATCTGG | MGLGRCIWEGWTLESE |
| peptide | GAAGGCTGGACCTTGGAGAGTGAG | ALRRDMGTWLLACICIC |
| | GCCCTGAGGCGAGACATGGGCACT | TCVCLGV |
| | GGCTCCTGGCCTGCATCTGCATCT | (SEQ ID NO: 12) |
| | GCACCTGTGTCTGCTTGGGAGTC | |
| | (SEQ ID NO: 11) | |
| IL15RA signal | ATGGCTCCTCGGAGAGCCAGAGGC | MAPRRARGCRTLGLPA |
| peptide | TGTAGAACACTTGGACTGCCCGCT | LLLLLLLRPPATRG |
| | CTGCTGCTGCTCCTGCTTCTTAGAC | (SEQ ID NO: 14) |
| | CTCCTGCCACAAGAGGC | |
| | (SEQ ID NO: 13) | |
| IL21R signal | ATGCCAAGAGGATGGGCCGCTCCT | MPRGWAAPLLLLLLQG |
| peptide | CTTCTCCTGTTGCTGCTTCAAGGCG | GWG (SEQ ID NO: 16) |
| | GCTGG GGC (SEQ ID NO: 15) | |
| IL2RA | GTGGCCGTGGCCGGATGTGTGTTT | VAVAGCVFLLISVLLLS |
| transmembrane | CTGCTGATCTCTGTGCTGCTGCTGA | GL (SEQ ID NO: 18) |
| domain | GCGGC CTG (SEQ ID NO: 17) | |
| IL2RB | ATCCCCTGGCTGGGACATCTGCTT | IPWLGHLLVGLSGAFGF |
| transmembrane | GTTGGACTGTCTGGCGCCTTCGGCT | IILVYLLI (SEQ ID NO: |
| domain | TCATCATCCTGGTGTATCTGCTGAT | 20) |
| | C (SEQ ID NO: 19) | |
| IL2RG | GTGGTCATCTCTGTGGGCTCTATGG | VVISVGSMGLIISLLCVY |
| transmembrane | GCCTGATCATCTCCCTGCTGTGTGT | FWL (SEQ ID NO: 22) |
| domain | GTACT TTTGGCTG (SEQ ID NO: 21) | |
| IL4RA | CTCCTGCTGGGCGTCAGCGTTTCCT | LLLGVSVSCIVILAVCLL |
| transmembrane | GCATTGTCATCCTGGCCGTCTGCCT | CYVSIT (SEQ ID NO: 24) |
| domain | GTTGTGCTATGTCAGCATCACC | |
| | (SEQ ID NO: 23) | |
| IL7RA | CCCATCCTGCTGACAATCAGCATCC | PILLTISILSFFSVALLVIL |
| transmembrane | TGAGCTTTTTCAGCGTGGCCCTGCT | ACVLW (SEQ ID NO: 26) |
| domain | GGTCATCCTGGCCTGTGTGCTGTGG | |
| | (SEQ ID NO: 25) | |
| IL9R | GGCAACACCCTTGTTGCTGTGTCC | GNTLVAVSIFLLLTGPT |
| transmembrane | ATCTTTCTCCTGCTGACTGGCCCGA | YLLF (SEQ ID NO: 28) |
| domain | CCTACCTCCTGTTC (SEQ ID NO: 27) | |
| IL15RA | GTGGCCATCAGCACAAGCACCGTT | VAISTSTVLLCGLSAVS |
| transmembrane | CTGCTGTGTGGCCTGTCAGCCGTTA | LLACYL (SEQ ID NO: 30) |
| domain | GCCTGC TGGCTTGCTACCTG | |
| | (SEQ ID NO: 29) | |
| IL21R | GGCTGGAATCCTCATCTGTTGTTGT | GWNPHLLLLLLLVIVFIP |
| transmembrane | TGCTCCTCCTGGTCATCGTGTTCAT | AFW (SEQ ID NO: 32) |
| domain | CCCCGC CTTTTGG (SEQ ID NO: 31) | |
| IL2RA | ACTTGGCAAAGACGGCAGAGAAA | TWQRRQRKSRRTI |
| intracellular | GAGCCGGCGGACCATC | (SEQ ID NO: 34) |
| domain | (SEQ ID NO: 33) | |
| IL2RB | AACTGCCGGAACACAGGCCCTTGG | NCRNTGPWLKKVLKCN |
| intracellular | CTGAAGAAAGTGCTGAAGTGCAAC | TPDPSKFFSQLSSEHGG |
| domain | ACCCCTGATCCGAGCAAGTTCTTTA | DVQKWLSSPFPSSSFSP |
| | GCCAGCTGAGCAGCGAGCATGGCG | GGLAPEISPLEVLERDK |
| | GCGACGTTCAGAAATGGCTGTCTA | VTQLLLQQDKVPEPASL |
| | GCCCATTTCCTAGCAGCAGCTTCAG | SSNHSLTSCFTNQGYFF |
| | CCCAGGTGGACTGGCCCCTGAGAT | FHLPDALEIEACQVYFT |
| | TAGCCCTCTGGAAGTGCTGGAACG | YDPYSEEDPDEGVAGA |
| | GGACAAAGTGACCCAGCTGCTCCT | PTGSSPQPLQPLSGEDD |
| | CCAGCAGGATAAGGTGCCAGAACC | AYCTFPSRDDLLLFSPSL |
| | TGCCAGCCTGTCCAGCAATCACAG | LGGPSPPSTAPGGSGAG |
| | CCTGACCAGCTGCTTTACCAACCA | EERMPPSLQERVPRDW |
| | GGGCTACTTCTTCTTCCATCTGCCT | DPQPLGPPTPGVPDLVD |
| | GACGCTCTGGAAATCGAGGCCTGC | FQPPPELVLREAGEEVP |
| | CAGGTGTACTTCACCTACGATCCCT | DAGPREGVSFPWSRPPG |
| | ACAGCGAAGAGGACCCCGATGAAG | QGEFRALNARLPLNTD |
| | GTGTTGCTGGCGCCCCTACAGGAT | AYLSLQELQGQDPTHL |
| | CTTCTCCACAGCCTCTGCAACCTCT | V (SEQ ID NO: 36) |
| | GAGCGGCGAGGATGATGCCTACTG | |
| | CACCTTTCCAAGCAGGGACGACCT | |
| | GCTCCTGTTCAGCCCATCTCTGCTC | |
| | GGAGGACCATCTCCTCCATCTACA | |
| | GCTCCAGGCGGATCTGGCGCTGGC | |
| | GAGGAAAGAATGCCACCTAGCCTG | |
| | CAAGAGCGGGTGCCCAGAGATTGG | |
| | GATCCTCAACCTCTCGGCCCTCCAA | |
| | CACCTGGCGTGCCAGATCTCGTGG | |
| | ACTTTCAGCCTCCTCCAGAGCTGGT | |
| | GCTGAGAGAAGCTGGCGAAGAAGT | |
| | GCCAGACGCTGGCCCTAGAGAGGG | |
| | CGTTAGCTTTCCTTGGAGCAGACCT | |
| | CCTGGACAGGGCGAGTTTAGGGCC | |
| | CTGAATGCAAGACTGCCTCTGAAC | |
| | ACCGACGCCTACCTGTCTCTGCAA | |
| | GAACTGCAGGGACAAGACCCCACA | |
| | CACCTGGTG (SEQ ID NO: 35) | |
| IL2RG | GAACGGACCATGCCTCGGATCCCC | ERTMPRIPTLKNLEDLV |
| intracellular | ACACTGAAGAACCTGGAAGATCTG | TEYHGNFSAWSGVSKG |
| domain | GTCACCGAGTACCACGGCAACTTC | LAESLQPDYSERLCLVS |
| | AGTGCTTGGAGCGGCGTGTCAAAA | EIPPKGGALGEGPGASP |
| | GGACTGGCCGAAAGCCTGCAGCCT | CNQHSPYWAPPCYTLK |
| | GACTACTCCGAGAGACTGTGCCTG | PET (SEQ ID NO: 38) |
| | GTGTCTGAGATCCCTCCTAAAGGC | |
| | GGCGCTCTCGGAGAAGGACCTGGT | |
| | GCCTCTCCATGCAATCAGCACAGC | |
| | CCTTATTGGGCCCCTCCTTGCTACA | |
| | CCCTGAAACCTGAGACA | |
| | (SEQ ID NO: 37) | |
| IL4RA | AAGATTAAGAAAGAATGGTGGGAT | KIKKEWWDQIPNPARSR |
| intracellular | CAGATTCCCAACCCAGCCCGCAGC | LVAIIIQDAQGSQWEKR |
| domain | CGCCTCGTGGCTATAATAATCCAG | SRGQEPAKCPHWKNCL |
| | GATGCTCAGGGGTCACAGTGGGAG | TKLLPCFLEHNMKRDE |
| | AAGCGGTCCCGAGGCCAGGAACCA | DPHKAAKEMPFQGSGK |
| | GCCAAGTGCCCACACTGGAAGAAT | SAWCPVEISKTVLWPESI |
| | TGTCTTACCAAGCTCTTGCCCTGTT | SVVRCVELFEAPVECEE |
| | TTCTGGAGCACAACATGAAAAGGG | EEEVEEEKGSFCASPESS |
| | ATGAAGATCCTCACAAGGCTGCCA | RDDFQEGREGIVARLTE |
| | AAGAGATGCCTTTCCAGGGCTCTG | SLFLDLLGEENGGFCQQ |
| | GAAAATCAGCATGGTGCCCAGTGG | DMGESCLLPPSGSTSAH |
| | AGATCAGCAAGACAGTCCTCTGGC | MPWDEFPSAGPKEAPP |
| | CAGAGAGCATCAGCGTGGTGCGAT | WGKEQPLHLEPSPPASP |
| | GTGTGGAGTTGTTTGAGGCCCCGG | TQSPDNLTCTETPLVIAG |
| | TGGAGTGTGAGGAGGAGGAGGAG | NPAYRSFSNSLSQSPCPR |
| | GTAGAGGAAGAAAAAGGGAGCTT | ELGPDPLLARHLEEVEP |
| | CTGTGCATCGCCTGAGAGCAGCAG | EMPCVPQLSEPTTVPQP |
| | GGATGACTTCCAGGAGGGAAGGG | EPETWEQILRRNVLQHG |
| | AGGGCATTGTGGCCCGGCTAACAG | AAAAPVSAPTSGYQEFV |
| | AGAGCCTGTTCCTGGACCTGCTCG | HAVEQGGTQASAVVGL |
| | GAGAGGAGAATGGGGGCTTTTGCC | GPPGEAGYKAFSSLLAS |
| | AGCAGGACATGGGGGAGTCATGCC | SAVSPEKCGFGASSGEE |
| | TTCTTCCACCTTCGGGAAGTACGA | GYKPFQDLIPGCPGDPA |
| | GTGCTCACATGCCCTGGGATGAGT | PVPVPLFTFGLDREPPRS |
| | TCCCAAGTGCAGGGCCCAAGGAGG | PQSSHLPSSSPEHLGLEP |
| | CACCTCCCTGGGGCAAGGAGCAGC | GEKVEDMPKPPLPQEQ |
| | CTCTCCACCTGGAGCCAAGTCCTC | ATDPLVDSLGSGIVYSA |
| | CTGCCAGCCCGACCCAGAGTCCAG | LTCHLCGHLKQCHGQE |
| | ACAACCTGACTTGCACAGAGACGC | DGGQTPVMASPCCGCC |
| | CCCTCGTCATCGCAGGCAACCCTG | CGDRSSPPTTPLRAPDPS |
| | CTTACCGCAGCTTCAGCAACTCCCT | PGGVPLEASLCPASLAPS |
| | GAGCCAGTCACCGTGTCCCAGAGA | GISEKSKSSSSFHPAPGN |
| | GCTGGGTCCAGACCCACTGCTGGC | AQSSSQTPKIVNFVSVGP |
| | CAGACACCTGGAGGAAGTAGAACC | TYMRVS |
| | CGAGATGCCCTGTGTCCCCCAGCT | (SEQ ID NO: 40) |
| | CTCTGAGCCAACCACTGTGCCCCA | |
| | ACCTGAGCCAGAAACCTGGGAGCA | |
| | GATCCTCCGCCGAAATGTCCTCCA | |
| | GCATGGGGCAGCTGCAGCCCCCGT | |
| | CTCGGCCCCCACCAGTGGCTATCA | |
| | GGAGTTTGTACATGCGGTGGAGCA | |
| | GGGTGGCACCCAGGCCAGTGCGGT | |
| | GGTGGGCTTGGGTCCCCCAGGAGA | |
| | GGCTGGTTACAAGGCCTTCTCAAG | |
| | CCTGCTTGCCAGCAGTGCTGTGTCC | |
| | CCAGAGAAATGTGGGTTTGGGGCT | |
| | AGCAGTGGGGAAGAGGGGTATAA | |
| | GCCTTTCCAAGACCTCATTCCTGGC | |
| | TGCCCTGGGGACCCTGCCCCAGTC | |
| | CCTGTCCCCTTGTTCACCTTTGGAC | |
| | TGGACAGGGAGCCACCTCGCAGTC | |
| | CGCAGAGCTCACATCTCCCAAGCA | |
| | GCTCCCCAGAGCACCTGGGTCTGG | |
| | AGCCGGGGGAAAAGGTAGAGGAC | |
| | ATGCCAAAGCCCCCACTTCCCCAG | |
| | GAGCAGGCCACAGACCCCCTTGTG | |
| | GACAGCCTGGGCAGTGGCATTGTC | |
| | TACTCAGCCCTTACCTGCCACCTGT | |
| | GCGGCCACCTGAAACAGTGTCATG | |
| | GCCAGGAGGATGGTGGCCAGACCC | |
| | CTGTCATGGCCAGTCCTTGCTGTGG | |
| | CTGCTGCTGTGGAGACAGGTCCTC | |
| | GCCCCCTACAACCCCCCTGAGGGC | |
| | CCCAGACCCCTCTCCAGGTGGGGT | |
| | TCCACTGGAGGCCAGTCTGTGTCC | |
| | GGCCTCCCTGGCACCCTCGGGCAT | |
| | CTCAGAGAAGAGTAAATCCTCATC | |
| | ATCCTTCCATCCTGCCCCTGGCAAT | |
| | GCTCAGAGCTCAAGCCAGACCCCC | |
| | AAAATCGTGAACTTTGTCTCCGTG | |
| | GGACCCACATACATGAGGGTCTCT | |
| | (SEQ ID NO: 39) | |
| IL 7RA | AAGAAGCGGATCAAGCCCATCGTG | KKRIKPIVWPSLPDHKK |
| intracellular | TGGCCCAGCCTGCCTGACCACAAG | TLEHLCKKPRKNLNVSF |
| domain | AAAACCCTGGAACACCTGTGCAAG | NPESFLDCQIHRVDDIQ |
| | AAGCCCCGGAAGAACCTGAACGTG | ARDEVEGFLQDTFPQQL |
| | TCCTTCAATCCCGAGAGCTTCCTGG | EESEKQRLGGDVQSPNC |
| | ACTGCCAGATCCACAGAGTGGACG | PSEDVVITPESFGRDSSL |
| | ACATCCAGGCCAGGGACGAAGTGG | TCLAGNVSACDAPILSSS |
| | AAGGCTTTCTGCAGGACACATTCC | RSLDCRESGKNGPHVY |
| | CTCAGCAGCTGGAAGAGAGCGAGA | QDLLLSLGTTNSTLPPPF |
| | AGCAGAGACTCGGAGGCGACGTGC | SLQSGILTLNPVAQGQPI |
| | AGAGCCCTAATTGCCCTTCTGAGG | LTSLGSNQEEAYVTMSS |
| | ACGTCGTGATCACCCCTGAGAGCT | FYQNQ (SEQ ID NO: 42) |
| | TCGGCAGAGATAGCAGCCTGACAT | |
| | GTCTGGCCGGCAATGTGTCCGCCT | |
| | GTGATGCCCCTATCCTGAGCAGCA | |
| | GCAGAAGCCTGGATTGCAGAGAGA | |
| | GCGGCAAGAACGGCCCTCACGTGT | |
| | ACCAGGATCTGCTCCTGAGCCTGG | |
| | GAACCACCAATAGCACACTGCCTC | |
| | CACCATTCAGCCTGCAGAGCGGCA | |
| | TCCTGACACTGAACCCTGTTGCTCA | |
| | GGGCCAGCCAATCCTGACCAGCCT | |
| | GGGCAGCAATCAAGAAGAGGCCTA | |
| | CGTCACCATGAGCAGCTTCTACCA | |
| | GAACCAG (SEQ ID NO: 41) | |
| IL9R | AAGCTGTCACCTAGAGTGAAGCGC | KLSPRVKRIFYQNVPSP |
| intracellular | ATATTCTATCAGAATGTGCCCAGC | AMFFQPLYSVHNGNFQ |
| domain | CCTGCGATGTTCTTCCAGCCATTGT | TWMGAHGAGVLLSQD |
| | ATAGCGTACACAATGGCAATTTCC | CAGTPQGALEPCVQEA |
| | AGACCTGGATGGGCGCCCACGGAG | TALLTCGPARPWKSVA |
| | CTGGTGTTTTGTTGTCTCAAGATTG | LEEEQEGPGTRLPGNLS |
| | TGCAGGGACGCCCCAAGGCGCGTT | SEDVLPAGCTEWRVQT |
| | GGAGCCTTGTGTCCAGGAAGCAAC | LAYLPQEDWAPTSLTRP |
| | CGCTCTTCTCACATGTGGCCCTGCT | APPDSEGSRSSSSSSSSN |
| | AGGCCATGGAAAAGCGTGGCTTTG | NNNYCALGCYGGWHL |
| | GAGGAGGAGCAAGAGGGCCCAGG | SALPGNTQSSGPIPALAC |
| | CACGAGACTTCCAGGAAACCTCTC | GLSCDHQGLETQQGVA |
| | CAGCGAGGACGTTCTGCCCGCTGG | WVLAGHCQRPGLHEDL |
| | ATGCACAGAATGGCGAGTGCAGAC | QGMLLPSVLSKARSWT |
| | GCTGGCTTATTTGCCTCAGGAGGA | F (SEQ ID NO: 44) |
| | CTGGGCCCCAACAAGCCTTACCAG | |
| | ACCCGCGCCACCAGATTCAGAGGG | |
| | AAGCCGATCCAGTTCTAGCTCCTC | |
| | ATCAAGCAACAATAACAATTATTG | |
| | CGCTCTGGGATGTTATGGTGGATG | |
| | GCACTTGAGCGCGTTGCCAGGCAA | |
| | CACGCAATCATCTGGTCCCATACC | |
| | CGCGCTTGCATGCGGACTGAGTTG | |
| | CGACCATCAGGGTCTCGAAACTCA | |
| | GCAAGGTGTTGCGTGGGTCCTGGC | |
| | GGGTCATTGCCAAAGACCTGGCCT | |
| | GCACGAGGATCTTCAGGGAATGCT | |
| | TTTGCCAAGTGTGCTCTCCAAGGCT | |
| | CGATCTTGGACGTTC | |
| | (SEQ ID NO: 43) | |
| IL15RA | AAGTCCAGACAGACACCTCCTCTG | KSRQTPPLASVEMEAM |
| intracellular | GCCAGCGTGGAAATGGAAGCCATG | EALPVTWGTSSRDEDLE |
| domain | GAAGCTCTGCCAGTGACCTGGGGC | NCSHHL |
| | ACCTCCAGCAGAGATGAGGATCTG | (SEQ ID NO: 46) |
| | GAAAACTGCAGCCACCACCTGTGA | |
| | TGA (SEQ ID NO: 45) | |
| IL21R | AGCCTGAAAACACACCCTCTGTGGC | SLKTHPLWRLWKKIWA |
| intracellular | GGCTGTGGAAGAAAATCTGGGCCG | VPSPERFFMPLYKGCSG |
| domain | TGCCATCTCCTGAGCGGTTCTTCAT | DFKKWVGAPFTGSSLEL |
| | GCCTCTGTACAAGGGCTGCAGCGG | GPWSPEVPSTLEVYSCH |
| | CGACTTCAAGAAATGGGTCGGAGC | PPRSPAKRLQLTELQEP |
| | CCCTTTTACCGGCAGCTCTCTGGAA | AELVESDGVPKPSFWPT |
| | CTTGGACCTTGGAGCCCTGAAGTGC | AQNSGGSAYSEERDRP |
| | CCAGCACACTGGAAGTGTACAGCT | YGLVSIDTVTVLDAEGP |
| | GTCACCCTCCTAGAAGCCCCGCCAA | CTWPCSCEDDGYPALD |
| | GAGACTGCAGCTCACAGAGCTGCA | LDAGLEPSPGLEDPLLD |
| | AGAGCCTGCCGAGCTGGTGGAATC | AGTTVLSCGCVSAGSPG |
| | TGATGGCGTGCCCAAGCCTAGCTTC | LGGPLGSLLDRLKPPLA |
| | TGGCCCACCGCTCAAAATTCTGGCG | DGEDWAGGLPWGGRSP |
| | GCAGCGCCTACAGCGAGGAAAGAG | GGVSESEAGSPLAGLD |
| | ATAGACCTTACGGCCTGGTGTCCAT | MDTFDSGFVGSDCSSPV |
| | CGACACCGTGACAGTGCTGGATGC | ECDFTSPGDEGPPRSYL |
| | CGAGGGACCTTGTACCTGGCCTTGT | RQWVVIPPPLSSPGPQA |
| | AGCTGCGAGGACGATGGCTACCCT | S (SEQ ID NO: 48) |
| | GCTCTGGATCTGGACGCAGGACTG | |
| | GAACCTTCTCCAGGCCTCGAAGATC | |
| | CTCTGCTGGACGCCGGAACAACAG | |
| | TGCTGTCTTGTGGCTGTGTGTCCGC | |
| | CGGATCTCCTGGACTTGGAGGACCT | |
| | CTGGGAAGCCTGCTGGACAGACTG | |
| | AAACCTCCTCTGGCCGATGGCGAA | |
| | GATTGGGCTGGTGGACTTCCTTGGG | |
| | GCGGAAGATCTCCAGGCGGAGTGT | |
| | CTGAATCTGAGGCCGGTTCTCCACT | |
| | GGCCGGCCTGGATATGGATACCTTC | |
| | GATAGCGGCTTCGTGGGCAGCGATT | |
| | GCAGCAGCCCTGTGGAATGCGACTT | |
| | CACATCTCCTGGCGACGAGGGCCC | |
| | ACCTAGAAGCTATCTCAGACAGTG | |
| | GTCGTGATCCCTCCACCTCTGTCTA | |
| | GTCCTGGACCACAGGCCAGC | |
| | (SEQ ID NO: 47) | |
| CD8 hinge | ACAACAACCCCTGCTCCTCGGCCTC | TTTPAPRPPTPAPTIASQ |
| | CTACACCAGCTCCTACAATTGCCA | PLSLRPEACRPAAGGAV |
| | GCCAGCCACTGTCTCTGAGGCCCG | HTRGLDFACD |
| | AAGCTTGTAGACCTGCTGCAGGCG | (SEQ ID NO: 50) |
| | GAGCCGTGCATACAAGAGGACTGG | |
| | ACTTCGCCTGTGAC | |
| | (SEQ ID NO: 49) | |
| anti-fluorescein | GACGTGGTCATGACACAGACCCCT | DVVMTQTPLSLPVSLGD |
| scFv 4M5.4 | CTGTCTCTGCCTGTGTCTCTGGGAG | QASISCRSSQSLVHSNG |
| | ATCAGGCCAGCATCAGCTGCAGAT | NTYLRWYLQKPGQSPK |
| | CTAGCCAGAGCCTGGTGCACAGCA | VLIYKVSNRVSGVPDRF |
| | ACGGCAACACCTACCTGCGGTGGT | SGSGSGTDFTLKINRVE |
| | ATCTGCAGAAGCCCGGCCAGTCTC | AEDLGVYFCSQSTHVP |
| | CTAAGGTGCTGATCTACAAGGTGT | WTFGGGTKLEIKSSADD |
| | CCAACAGAGTGTCCGGCGTGCCCG | AKKDAAKKDDAKKDD |
| | ATAGATTTTCTGGCAGCGGCTCTG | AKKDGGVKLDETGGGL |
| | GCACCGACTTCACCCTGAAGATCA | VQPGGAMKLSCVTSGF |
| | ATAGAGTGGAAGCCGAGGACCTGG | TFGHYWMNWVRQSPE |
| | GCGTGTACTTCTGTAGCCAGTCTAC | KGLEWVAQFRNKPYNY |
| | CCACGTGCCATGGACCTTTGGCGG | ETYYSDSVKGRFTISRD |
| | CGGAACAAAGCTGGAAATCAAGA | DSKSSVYLQMNNLRVE |
| | GCAGCGCCGACGACGCCAAGAAG | DTGIYYCTGASYGMEY |
| | GACGCCGCTAAGAAGGATGACGCC | LGQGTSVTVS |
| | AAAAAAGACGATGCCAAAAAGGA | (SEQ ID NO: 52) |
| | TGGCGGCGTGAAGCTGGACGAAAC | |
| | AGGCGGAGGACTTGTTCAGCCTGG | |
| | CGGAGCCATGAAGCTGAGCTGTGT | |
| | GACCAGCGGCTTCACCTTCGGCCA | |
| | CTACTGGATGAACTGGGTCCGACA | |
| | GAGCCCTGAGAAAGGCCTGGAATG | |
| | GGTCGCCCAGTTCAGAAACAAGCC | |
| | CTACAACTACGAAACCTACTACAG | |
| | CGACAGCGTGAAGGGCAGATTCAC | |
| | CATCAGCCGGGACGACAGCAAGTC | |
| | CAGCGTGTACCTGCAGATGAACAA | |
| | CCTGCGCGTGGAAGATACCGGCAT | |
| | CTACTACTGTACCGGCGCCAGCTA | |
| | CGGCATGGAATATCTCGGCCAGGG | |
| | CACCAGCGTGACCGTGTCT | |
| | (SEQ ID NO: 51) | |
| anti-MPOB scFv | CAAGTGCGGCTGCAAGAGTCTGGA | QVRLQESGPSLVKPSQT |
| | CCTAGCCTGGTCAAGCCCAGCCAG | LSLTCTVSGFSLTNYYV |
| | ACACTGAGCCTGACCTGTACCGTG | GWVRQAPGKALEWVG |
| | TCCGGCTTCAGCCTGACCAACTAC | VMDSGGGTYYNPALKS |
| | TATGTCGGCTGGGTCCGACAGGCC | RLSITRDTSKSQVSLSLS |
| | CCTGGAAAAGCTCTTGAGTGGGTC | SVTTEDTAVYYCASFSF |
| | GGAGTGATGGATAGCGGCGGAGG | GRDWNYWGPGLLLTIP |
| | CACCTACTACAACCCCGCTCTGAA | SEGKSSGSGSESKVDSY |
| | AAGCCGGCTGAGCATCACCAGAGA | ELTQPSSVSRSLGQSVSI |
| | CACCAGCAAGTCTCAGGTGTCCCT | TCSGSSSNVGYGNYVG |
| | GAGCCTGTCTAGCGTGACCACAGA | WFQQVPGSAPKLLIYD |
| | GGATACCGCCGTGTACTACTGCGC | ATSRASGVPDRFSGSRS |
| | CAGCTTCAGCTTCGGCAGAGACTG | SNTATLTISSLQAEDEA |
| | GAATTATTGGGGCCCTGGCCTGCT | DYYCAHWDSSANIAIFG |
| | GCTGACAATCCCTTCTGAGGGCAA | SGSRLTVLG |
| | GTCTAGCGGCAGCGGCTCTGAGAG | (SEQ ID NO: 54) |
| | CAAGGTGGACAGCTATGAGCTGAC | |
| | CCAGCCTAGCAGCGTGTCCAGATC | |
| | TCTGGGCCAGTCCGTGTCCATCAC | |
| | CTGTAGCGGAAGCAGCAGCAATGT | |
| | CGGCTACGGCAATTACGTCGGCTG | |
| | GTTCCAGCAGGTCCCAGGCTCTGC | |
| | TCCTAAGCTGCTGATCTACGACGC | |
| | CACCTCTAGAGCCAGCGGCGTGCC | |
| | AGATAGATTCAGCGGCAGCAGAAG | |
| | CAGCAACACCGCCACACTGACAAT | |
| | CAGCAGTCTGCAGGCCGAGGACGA | |
| | GGCCGATTACTATTGTGCCCACTG | |
| | GGACAGCAGCGCCAATATCGCCAT | |
| | CTTTGGCTCCGGCAGCAGGCTGAC | |
| | AGTTCTGGGA (SEQ ID NO: 53) | |
| anti- | CAAGTTCGGCTGCAAGGCTCTGGA | QVRLQGSGPSLVKPSQT |
| Anthraquione | CCTAGCCTGGTCAAGCCTAGCCAG | LSLTCTVSGFSLTSNAV |
| scFv | ACACTGAGCCTGACCTGTACCGTG | DWVRQAPGKVPEWLG |
| | TCCGGCTTCAGCCTGACAAGCAAC | FIRGGGSTFYNSALKSR |
| | GCTGTGGACTGGGTCCGACAGGCT | LSITRDTSKSQVSLSLSS |
| | CCTGGAAAAGTGCCTGAGTGGCTG | VTTEDTAVYYCARASC |
| | GGCTTCATCAGAGGCGGCGGAAGC | SGDIYTDTCGIDYWGPG |
| | ACCTTCTACAACAGCGCCCTGAAG | LLVTVSSEGKSSGSGSE |
| | TCCAGACTGAGCATCACCAGAGAC | SKVDQSALTQPSSVSRS |
| | ACCAGCAAGAGCCAGGTGTCCCTG | LGQSVSITCSGSSSNVG |
| | AGCCTGTCTAGCGTGACCACAGAG | AGNYVNWFRLIPGSAP |
| | GACACCGCCGTGTACTACTGTGCC | KSLIYAATTRASGVPDR |
| | AGAGCCAGCTGTAGCGGCGACATC | FSGSRSGNTATLTISSLQ |
| | TACACAGACACCTGTGGCATCGAC | AEDEADYYCSSYDITAV |
| | TACTGGGGCCCTGGACTGCTGGTT | NLFGSGTRLTVLG |
| | ACCGTTAGCTCTGAGGGCAAGTCT | (SEQ ID NO: 56) |
| | AGCGGCAGCGGAAGCGAGAGCAA | |
| | AGTGGACCAGTCTGCCCTGACACA | |
| | GCCTAGCAGCGTGTCCAGATCTCT | |
| | GGGCCAGTCCGTGTCCATCACCTG | |
| | TTCTGGCAGCAGCTCTAACGTCGG | |
| | CGCTGGCAACTACGTGAACTGGTT | |
| | CAGACTGATCCCTGGCAGCGCCCC | |
| | TAAGAGCCTGATCTACGCTGCCAC | |
| | AACAAGAGCCTCTGGCGTGCCCGA | |
| | TAGATTCAGCGGCTCTAGAAGCGG | |
| | CAACACCGCCACACTGACAATCAG | |
| | CAGTCTGCAGGCCGAGGACGAGGC | |
| | CGACTACTACTGTAGCAGCTACGA | |
| | CATCACAGCCGTGAACCTGTTCGG | |
| | CTCCGGCACCAGACTGACAGTTCT | |
| | TGGA (SEQ ID NO: 55) | |
| anti-DOTA scFv | GCCTCTCACGTGAAGCTGCAAGAG | ASHVKLQESGPGLVQPS |
| | TCTGGACCTGGCCTGGTGCAGCCT | QSLSLTCTVSGFSLTDY |
| | AGCCAAAGCCTGTCTCTGACCTGT | GVHWVRQSPGKGLEW |
| | ACCGTGTCCGGCTTCAGCCTGACA | LGVIWSGGGTAYNTALI |
| | GATTACGGCGTGCACTGGGTCCGA | SRLNIYRDNSKNQVFLE |
| | CAGAGCCCTGGAAAAGGACTGGA | MNSLQAEDTAMYYCA |
| | ATGGCTGGGAGTGATTTGGAGCGG | RRGSYPYNYFDAWGCG |
| | CGGAGGCACAGCCTATAACACAGC | TTVTVSSGGGGSGGGG |
| | CCTGATCAGCAGACTGAACATCTA | SGGGGSQAVVIQESALT |
| | CCGGGACAACAGCAAGAACCAGG | TPPGETVTLTCGSSTGA |
| | TGTTCCTGGAAATGAACTCCCTGC | VTASNYANWVQEKPD |
| | AGGCCGAGGACACCGCCATGTACT | HCFTGLIGGHNNRPPGV |
| | ACTGTGCCAGAAGAGGCAGCTACC | PARFSGSLIGDKAALTIA |
| | CCTACAACTACTTCGACGCCTGGG | GTQTEDEAIYFCALWYS |
| | GCTGTGGCACCACCGTGACAGTTT | DHWVIGGGTRLTVLGG |
| | CTAGCGGAGGCGGAGGATCTGGTG | SEQKLISEEDL |
| | GCGGAGGTAGTGGTGGCGGTGGAT | (SEQ ID NO: 58) |
| | CTCAGGCCGTGGTCATCCAAGAAA | |
| | GCGCCCTGACAACACCTCCTGGCG | |
| | AGACAGTGACACTGACCTGTGGAT | |
| | CTTCTACAGGCGCCGTGACCGCCA | |
| | GCAACTACGCCAATTGGGTGCAAG | |
| | AGAAGCCCGACCACTGCTTCACAG | |
| | GCCTGATCGGCGGCCACAACAATA | |
| | GACCTCCAGGCGTGCCAGCCAGAT | |
| | TCAGCGGATCTCTGATCGGAGACA | |
| | AGGCCGCTCTGACAATCGCCGGCA | |
| | CACAGACAGAGGACGAGGCCATCT | |
| | ACTTTTGCGCCCTGTGGTACAGCG | |
| | ACCACTGGGTTATCGGCGGAGGAA | |
| | CCAGACTGACAGTGCTCGGCGGAT | |
| | CTGAGCAGAAGCTGATCTCCGAAG | |
| | AGGACCTG (SEQ ID NO: 57) | |

The signal peptide directs the nascent protein into the endoplasmic reticulum. In some embodiments, the signal peptide contained in the cytokine receptor switch is native to or derived from an interleukin-2 receptor alpha chain (IL-2RA), IL-2RB, IL-2RG, IL-4RA, IL-7RA, IL-9R, IL-15RA, or IL-21R. In some embodiments, the signal peptide is native to CD8, i.e., a CD8 signal peptide. In some embodiments, the signal peptide and the intracellular domain are native to the same cytokine receptor. In some embodiments, the signal peptide and the intracellular domain are native to different cytokine receptors.

In some embodiments, the signal peptide is native to IL-2RA and has nucleic acid sequence SEQ ID NO: 1 and amino acid sequence SEQ ID NO: 2.

In some embodiments, the signal peptide is native to IL-2RB and has nucleic acid sequence SEQ ID NO: 3 and amino acid sequence SEQ ID NO: 4.

In some embodiments, the signal peptide is native to IL-2RG and has nucleic acid sequence SEQ ID NO: 5 and amino acid sequence SEQ ID NO: 6.

In some embodiments, the signal peptide is native to IL-4RA and has nucleic acid sequence SEQ ID NO: 7 and amino acid sequence SEQ ID NO: 8.

In some embodiments, the signal peptide is native to IL-7RA and has nucleic acid sequence SEQ ID NO: 9 and amino acid sequence SEQ ID NO: 10.

In some embodiments, the signal peptide is native to IL-9R and has nucleic acid sequence SEQ ID NO: 11 and amino acid sequence SEQ ID NO: 12.

In some embodiments, the signal peptide is native to IL-15RA and has nucleic acid sequence SEQ ID NO: 13 and amino acid sequence SEQ ID NO: 14.

In some embodiments, the signal peptide is native to IL-21R and has nucleic acid sequence SEQ ID NO: 15 and amino acid sequence SEQ ID NO: 16.

The scFy binds a synthetic small molecule. The term “synthetic small molecule” as used herein refers to an organic molecule or compound that is monofuctional and that ranges in size from about 50 to about 10,000 daltons, usually from about 50 to about 5,000 daltons and more usually from about 100 to about 1000 daltons.

Representative examples of synthetic small molecules include fluorescein and fluorescein derivatives (e.g., FITC, 5-carboxyfluorescein, 6-carboxyfluorescein, 5/6-carboxyfluorescein, NHS-fluorescein (5(6)-Carboxyfluorescein N-hydroxysuccinimide ester), 5-(iodoacetamido) fluorescein, 5-([4,6-dichlorotriazin-2-yl] amino) fluorescein hydrochloride, 5-(bromomethyl) fluorescein, and fluorescein 5-carbamoylmethylthiopropanoic acid), 4-[(6-methylpyrazin-2-yl) oxy] benzoate (MPOB), anthraquinone-2-carboxylate (AQ), and tetraxetan (DOTA). In some embodiments, the synthetic small molecule is polymeric, wherein the polymer is a monopolymer, a heteropolymer, or a branched polymer. A representative example of a polymeric synthetic small molecule is a polyhistidine-tag (His-tag), e.g., having from about 6 to about 9) histidine (His) residues. An exemplary 6 His-tag has a molecular weight of about 800 daltons.

In some embodiments, the scFv binds a synthetic small molecule which is fluorescein and fluorescein derivatives, 4-[(6-methylpyrazin-2-yl) oxy] benzoate (MPOB), anthraquinone-2-carboxylate (AQ), tetraxetan (DOTA), a polyhistidine-tag (His-tag).

The synethetic small molecule is substantially nonimmunogenic. In some embodiments, the synthetic small molecule is nonimmunogenic such that if injected by itsself into an animal, it would not cause that animal to produce antibodies or T cells reactive thereto. In some embodiments, the synthetic small molecule generates IgM antibodies in an animal but does not cause antibody class switching. In some embodiments, the synthetic small molecule generates a low level of antibodies in an animal such that the synthetic small molecule may still bind one or more cytokine receptor switches without being neutralized. In some embodiments, the synthetic small molecule does not generate a significant immune response. A “significant immune” response is any immune response that would limit or restrict the in vivo utility of the synthetic small molecule as used in accordance with the teachings of the present disclosure.

A representative example of an scFv that binds fluorescein has nucleic acid sequence SEQ ID NO: 51 and amino acid sequence SEQ ID NO: 52.

A representative example of an scFv that binds MPOB has nucleic acid sequence SEQ ID NO: 53 and amino acid sequence SEQ ID NO: 54.

A representative example of an scFv that binds AQ has nucleic acid sequence SEQ ID NO: 55 and amino acid sequence SEQ ID NO: 56.

A representative example of an scFv that binds DOTA has nucleic acid sequence SEQ ID NO: 57 and amino acid sequence SEQ ID NO: 58.

The transmembrane (TM) domain allows the cytokine receptor switch to be stably anchored into the cell membrane of the immune cell. The transmembrane domain may be derived from the same protein or from a different protein from which the other domains of the cytokine receptor switch are derived. The transmembrane domain may be derived from a natural or from a recombinant source. Where the source is natural, the domain may be derived from any membrane-bound or transmembrane protein.

In some embodiments, the transmembrane domain is derived from IL-2RA, IL-2RB, IL-2RG, IL-4RA, IL-7RA, IL-9RA, IL-15RA, or IL-21R.

In some embodiments, the transmembrane domain peptide is derived from IL-2RA and has nucleic acid sequence SEQ ID NO: 17 and amino acid sequence SEQ ID NO: 18.

In some embodiments, the transmembrane domain peptide is derived from IL-2RB and has nucleic acid sequence SEQ ID NO: 19 and amino acid sequence SEQ ID NO: 20.

In some embodiments, the transmembrane domain peptide is derived from IL-2RG and has nucleic acid sequence SEQ ID NO: 21 and amino acid sequence SEQ ID NO: 22.

In some embodiments, the transmembrane domain peptide is derived from IL-4RA and has nucleic acid sequence SEQ ID NO: 23 and amino acid sequence SEQ ID NO: 24.

In some embodiments, the transmembrane domain peptide is derived from IL-7RA and has nucleic acid sequence SEQ ID NO: 25 and amino acid sequence SEQ ID NO: 26.

In some embodiments, the transmembrane domain peptide is derived from IL-9R and has nucleic acid sequence SEQ ID NO: 27 and amino acid sequence SEQ ID NO: 28.

In some embodiments, the transmembrane domain peptide is derived from IL-15RA and has nucleic acid sequence SEQ ID NO: 29 and amino acid sequence SEQ ID NO: 30.

In some embodiments, the transmembrane domain peptide is derived from IL-21R and has nucleic acid sequence SEQ ID NO: 31 and amino acid sequence SEQ ID NO: 32.

The cytokine receptor switch can be designed to include a transmembrane domain that is indirectly attached to the scFv. In such embodiments, the transmembrane domain is attached to the scFy via a hinge domain. As used herein, the term “hinge domain” refers to a domain that links the extracellular binding domain to the transmembrane domain, and may confer flexibility to the extracellular binding domain. In some embodiments, the hinge domain positions the extracellular domain close to the plasma membrane of the immune cell to minimize the potential for recognition by antibodies or binding fragments thereof. The hinge domain may be natural (such as a hinge from a human protein) or synthetic. Sources of hinge domains include human Ig (immunoglobulin) hinges (e.g., an IgG4 hinge, an IgD hinge), and a CD8 (e.g., CD8α hinge).

In some embodiments, the hinge domain is derived from cluster of differentiation 8 (CD8).

In some embodiments, the hinge domain peptide has nucleic acid sequence SEQ ID NO: 49 and amino acid sequence SEQ ID NO: 50.

As used herein, the term “intracellular domain” refers to a signaling moiety that provides to immune cells, such as T-cells, a signal which mediates a cellular response such as, for example, activation, proliferation, differentiation, and/or cytokine secretion. In some embodiments, the intracellular domain is native to or derived from IL-2RA, IL-2RB, IL-2RG, IL-4RA, IL-7RA, IL-9RA, IL-15RA, or IL-21R.

In some embodiments, the intracellular domain is derived from IL-2RA and has nucleic acid sequence SEQ ID NO: 33 and amino acid sequence SEQ ID NO: 34.

In some embodiments, the intracellular domain is derived from IL-2RB and has nucleic acid sequence SEQ ID NO: 35 and amino acid sequence SEQ ID NO: 36.

In some embodiments, the transmembrane domain peptide is derived from IL-2RG and has nucleic acid sequence SEQ ID NO: 37 and amino acid sequence SEQ ID NO: 38.

In some embodiments, the transmembrane domain peptide is derived from IL-4RA and has nucleic acid sequence SEQ ID NO: 39 and amino acid sequence SEQ ID NO: 40.

In some embodiments, the transmembrane domain peptide is derived from IL-7RA and has nucleic acid sequence SEQ ID NO: 41 and amino acid sequence SEQ ID NO: 42.

In some embodiments, the transmembrane domain peptide is derived from IL-9R and has nucleic acid sequence SEQ ID NO: 43 and amino acid sequence SEQ ID NO: 44.

In some embodiments, the transmembrane domain peptide is derived from IL-15RA and has nucleic acid sequence SEQ ID NO: 45 and amino acid sequence SEQ ID NO: 46.

In some embodiments, the transmembrane domain peptide is derived from IL-21R and has nucleic acid sequence SEQ ID NO: 47 and amino acid sequence SEQ ID NO: 48.

Representative cytokine receptor switches include combinations of the sequences of the signal peptides, scFvs, transmembrane domains, hinge domains and intracellular domains disclosed above.

In some embodiments, the cytokine receptor switch comprises an IL-2RB signal peptide, an anti-small molecule scFv, a CD8 hinge, an IL-2RB transmembrane domain, and an IL-2RB intracellular domain. In some embodiments, the cytokine receptor switch comprises an IL-2RG signal peptide, an anti-small molecule scFv, a CD8 hinge, an IL-2RG transmembrane domain, an IL-2RG intracellular domain. In some embodiments, the cytokine receptor switch comprises an IL-7RA signal peptide, an anti-small molecule scFv, a CD8 hinge, an IL-7RA transmembrane domain, and an IL-7RA intracellular domain. In some embodiments, the cytokine receptor switch comprises an IL-15RA signal peptide, an anti-small molecule scFv, a CD8 hinge, an IL-15RA transmembrane domain, and an IL-15RA intracellular domain.

In some embodiments, the cytokine receptor switch of the present disclosure is an anti-fluorescein-IL2-RA cytokine receptor switch and has an amino acid sequence (SEQ ID NO: 59):

| | |
|---|---|
| 1 | mdsyllmwgl ltfimvpgcq advvmtqtpl slpvslgdqa siscrssqsl vhsngntylr |
| 61 | wylakpgqsp kvliykvsnr vsgvpdrfsg sgsgtdftlk inrveaedlg vyfcsqsthv |
| 121 | pwtfgggtkl eikssaddak kdaakkddak kddakkdggv kldetggglv qpggamklsc |
| 181 | vtsgftfghy wmnwvrqspe kglewvaqfr nkpynyetyy sdsvkgrfti srddskssvy |
| 241 | lqmnnlrved tgiyyctgas ygmeylgqgt svtvstttpa prpptpapti asqplslrpe |
| 301 | acrpaaggav htrgldfacd vavagcvfll isvlllsglt wqrrqksrr ti |

### Immune Cells Comprising Nucleic Acids Encoding Inventive Cytokine Receptor Switches

In some aspects, the present disclosure is directed to a composition comprising an immune cell comprising at least one nucleic acid encoding a cytokine receptor switch. Immune cells useful in the present disclosure are mammalian, preferably primate immune cells such as immune cells from monkeys, and humans. In some embodiments, the immune cells are T cells. In some embodiments, the immune cells are NK cells. In some embodiments, the immune cells are allogeneic (from the same species but different donor) as the recipient subject: in some embodiments the immune cells are autologous (the donor and the recipient are the same): in some embodiments the immune cells are syngeneic (the donor and the recipients are different but are identical twins). Natural killer (NK) cells are an important effector cell type for adoptive cancer immunotherapy. Similar to T cells, in some embodiments, the NK cells useful in the present disclosure are allogeneic, autologous, or syngeneic. For example, in some embodiments, the T cells are CD8⁺ or CD4⁺ T cells. In some embodiments, NK cells are CD56^{dim} CD16⁺ NK cells. In some embodiments, NK cells are CD56^{bright} CD16⁻ NK cells. In some embodiments, the NK cells are primary NK cells, memory-like NK cells, or induced memory like NK cells. The compositions may include combinations of two or more types of immune cells that comprise the same or different cytokine receptor switch encoded by a nucleic acid.

In some embodiments, the composition comprises an immune cell that comprises a nucleic acid that encodes the anti-fluorescein-IL2-RA cytokine receptor switch having the amino acid sequence SEQ ID NO: 59.

In some embodiments, the immune cells comprise at least two nucleic acids that encode at least two cytokine receptor switches wherein at least one of the respective signal peptides, transmembrane domains, and intracellular domains are different.

For example, in some embodiments, the at least two cytokine receptor switches comprise different scFvs.

In some embodiments, the immune cells comprise at least two nucleic acids that encode at least two cytokine receptor switches, the nucleic acids encoding a first cytokine switch comprising an IL-2RG signal peptide, an anti-small molecule scFv, a CD8 hinge, an IL-2RG transmembrane domain, an IL-2RG intracellular domain, and a second cytokine switch comprising an IL-7RA signal peptide, an anti-small molecule scFv, a CD8 hinge, an IL-7RA transmembrane domain, and an IL-7RA intracellular domain.

In some embodiments, the immune cells comprise at least three nucleic acids that encodes at least three cytokine receptor switches wherein at least one of the respective signal peptides, transmembrane domains, and intracellular domains are different.

In some embodiments, the immune cells comprise at least three nucleic acids that encode at least three cytokine receptor switches comprising a first cytokine switch comprising an IL-2RB signal peptide, an anti-small molecule scFv, a CD8 hinge, an IL-2RB transmembrane domain, and an IL-2RB intracellular domain, a second cytokine switch comprising an IL-2RG signal peptide, an anti-small molecule scFv, a CD8 hinge, an IL-2RG transmembrane domain, and an IL-2RG intracellular domain, and a third cytokine switch comprising an IL-15RA signal peptide, an anti-small molecule scFv, a CD8 hinge, an IL-15RA transmembrane domain, and an IL-15RA intracellular domain.

In some embodiments, the immune cells are derived from induced pluripotent stem cells (iPSC), cord blood, or peripheral blood mononuclear cells (PBMCs). Precursor harvesting, generation, and maintenance of iPSC are known in the art. See, for example, U.S. Pat. Nos.: 9,260,696, 10,214,722, 10,428,309, 10,844,356, and 11,193,108. Similar to iPSC, methods for harvesting, generating, and maintenaning stem cells from cord blood are known in the art. See, U.S. Pat. Nos.: 6,338,942, 7,311,905, 8,889,411 and 9,260,696. Similarly, methods for harvesting, generating, and maintenaning immune cells from PBMCs are known in the art. See, U.S. Pat. Nos. 9,476,028, 11, 162,072, 11,229,689 and U.S. Patent Publication No. 2017/0051252. Methods for differentiating and isolating T and NK cells from progenitor, pluripotent, or stem cells into a desired cell subset are known in the art.

### Chimeric Antigen Receptor (CAR)-Immune Cells

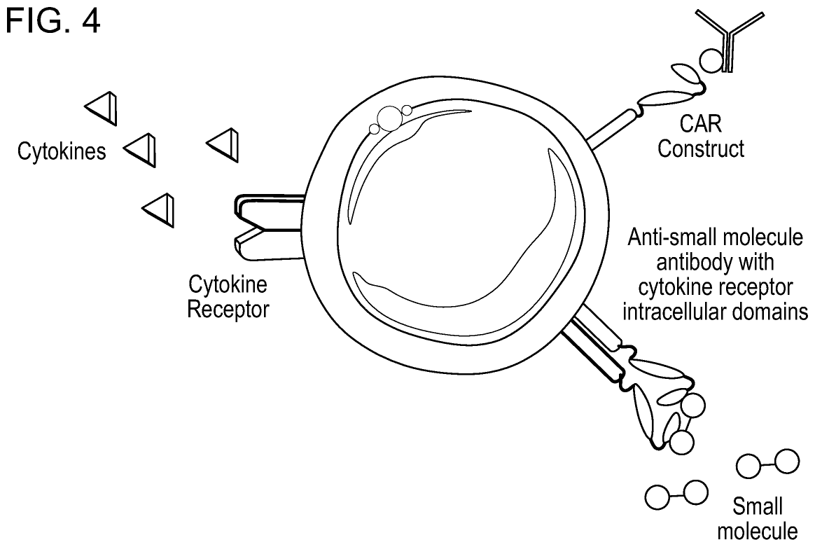

In some embodiments, the immune cells are chimeric antigen receptor (CAR)-immune cells (e.g., CAR-T cells) and also contain a CAR directed against a cell surface antigen (FIG. 1B, FIG. 4).

The term “Chimeric antigen receptor” (CAR) as used herein refers to a synthetically designed receptor comprising an extracellular binding domain that includes an antibody or binding fragment thereof, nanobody or other protein sequence that binds to a cell antigen associated with a disease or disorder (a “cell associated antigen”) and is linked via a spacer domain to an intracellular domain of a T cell. The spacer domain includes a transmembrane domain, and in some embodiments, a hinge domain.

### Extracellular Binding Domain

As used herein, an extracellular binding domain is a moiety that specifically binds a target antigen, namely a cell surface antigen such as a tumor associated antigen. Extracellular binding domains may include a protein, polypeptide, oligopeptide, or peptide. The extracellular binding domain may be naturally occurring, synthetic, semi-synthetic, or recombinantly produced.

In some embodiments, the extracellular binding domain binds a cell associated antigen on a tumor cell (a “tumor associated antigen” (TAA)).

In some embodiments, the extracellular binding domain of the CAR is a single-chain variable fragment (scFv) of an antibody (as defined above).

Other types of antibody fragments having specificity for cell associated antigens that may be useful as components of the CAR include Fv, Fab, and 'Fab′)₂ fragments. See, e.g., U.S. Pat. No. 4,946,788.

Representative examples of such extracellular binding domains are set forth in Table 2:

TABLE 2

| TAA | Targeting Moiety | Source |
|---|---|---|
| ACVR1 | mAb C-5 | Santa Cruz Biotechnology |
| ALK | Antibody or fragment thereof | Mino-Kenudson et al., Clin. Cancer |
| | | Res. 16(5): 1561-1571 (2010) |
| B7H3 (also known | MGA271 | Macrogenics |
| as CD276) | | |
| BCMA | Antibody or fragment thereof, | Friedman et al., Hum. Gene Ther. |
| | Belantamab (anti-BCMA | 29(5): 585-601 (2018), Raje et al., N |
| | antibody conjugated with | Engl. J. Med., 380(18): 1726-1737 |
| | MMAF mitotic agent), anti- | (2019), WO2010104949A2 and |
| | BCMA antibody J6M0 | WO2003014294A2, Abeomics |
| | (belantamab biosimilar). | |
| BST2 (also known | Anti-CD317 antibody, | Antibodies-Online |
| as CD317) | Monoclonal [3H4] | |
| BST2 (also known | Anti-CD317 antibody, | R&D Systems |
| as CD317) | Monoclonal[696739] | |
| CAIX | antibody clone 303123 | R&D Systems |
| CEA | Antibody or fragment thereof | Chmielewski et al., Gastoenterology |
| | | 143(4): 1095-1107 (2012) |
| CLDN6 | antibody IMAB027 | Ganymed Pharmaceuticals; |
| | | clinicaltrial.gov/show/NCT02054351 |
| CS1 | Antibody or fragment thereof, | WO2004100898A2 |
| | Elotuzumab | |
| CYP1B1 | Antibody or fragment thereof | Maecker et al., Blood 102(9): 3287- |
| | | 3294 (2003) |
| EGFR | cetuximab, panitumumab, | Commercial sources |
| | zalutumumab, nimotuzumab, | |
| | matuzumab | |
| EPCAM | MT110, EpCAM-CD3 | clinicaltrials.gov/ct2/show/NCT0063 |
| | bispecific Ab | 5596) |
| EPCAM | Edrecolomab; 3622W94; ING- | Commercial sources |
| | 1; and adecatumumab (MT201) | |
| EphA2 | Antibody or fragment thereof | Yu et al., Mol. Ther. 22(1): 102-111 |
| | | (2014) |
| Ephrin B2 | Antibody or fragment thereof | Abengozar et al., Blood |
| | | 119(19): 4565-4576 (2012) |
| ERBB2 | trastuzumab, or pertuzumab | Commercial sources |
| (HER2/neu) | | |
| FAP | Antibody or fragment thereof | Ostermann et al., Clinical Cancer |
| | | Research 14: 4584-4592 (2008) |
| FAP | sibrotuzumab | Hofheinz et al., Oncology Research |
| | | and Treatment 26: 44-48 (2003); and |
| | | Tran et al., J. Exp. Med. |
| | | 210(6): 1125-1135 (2013) |
| FCAR | CD89/FCAR Antibody | Sino Biological Inc. |
| | (Catalog#10414-H08H) | |
| Folate receptor | Antibody IMGN853 | Commercial sources |
| alpha | | |
| Folate receptor | Antibody or fragment thereof | U.S. Patent Application Publication |
| alpha | | 20120009181; U.S. Pat. No. 4,851,332 |
| | | and U.S. Pat. No. 5,952,484 |
| Fos-related antigen | Antibody 12F9 | Novus Biologicals |
| 1 | | |
| Fucosy1 GM1 | Antibody or fragment thereof | U.S. Patent Application Publication |
| | | 20100297138; WO2007067992 |
| GD2 | Antibody or fragment thereof | Mujoo et al., Cancer Res. |
| | | 47(4): 1098-1104 (1987); Cheung et |
| | | al., Cancer Res. 45(6): 2642-2649 |
| | | (1985), Cheung et al., J. Clin. Oncol. |
| | | 5(9): 1430-1440 (1987), Cheung et |
| | | al., J. Clin. Oncol. 16(9): 3053-3060 |
| | | (1998), Handgretinger et al., Cancer |
| | | Immunol. Immunother. 35(3): 199- |
| | | 204 (1992); U.S. Publication No.: |
| | | 20100150910; WO2011160119 |
| GD2 | mAb 14.18, 14G2a, ch14.18, | WO2012033885, WO2013040371, |
| | hul4.18, 3F8, hu3F8, 3G6, 8B6, | WO2013192294, WO2013061273, |
| | 60C3, 10B8, ME36.1, and 8H9; | WO2013123061, WO2013074916, |
| | or fragments thereof | and WO201385552 |
| GD3 | Antibody or fragment thereof | U.S. Pat. No. 7,253,263; U.S. Pat. No. |
| | | 8,207,308; U.S. Patent Application |
| | | Publication 20120276046; |
| | | EP1013761; WO2005035577; and |
| | | U.S. Pat. No. 6,437,098 |
| GD3 | Antibody or fragment thereof | U.S. Pat. No. 7,253,263; U.S. Pat. No. |
| | | 8,207,308; U.S. Patent Application |
| | | Publication 20120276046; |
| | | EP1013761 A3; U.S. Patent |
| | | Application Publication |
| | | 20120276046; WO2005035577; U.S. |
| | | Pat. No. 6,437,098 |
| GloboH | Antibody or fragment thereof | Kudryashov et al., Glycoconj J. |
| | | 15(3): 243-9 (1998), Lou et al., Proc. |
| | | Natl. Acad. Sci. USA. 111(7): 2482- |
| | | 2487 (2014) |
| GloboH | Antibody MBr1 | Bremer et al., J. Biol. Chem. |
| | | 259: 14773-14777 (1984) |
| GM3 | antibody CA 2523449 (mAb | Commercial sources |
| | 14F7) | |
| Gp100 | antibody HMB45, NKIbetaB | Commercial sources |
| Gp100 | Antibody or fragment thereof | WO2013165940; U.S. Patent |
| | | Application Publication |
| | | 20130295007 |
| GPC3 | Antibody hGC33 | Nakano et al., Anticancer Drugs |
| | | 21(10): 907-916 (2010) |
| GPC3 | Antibody MDX-1414, HN3, | Feng et al., FEBS Lett. 588(2): 377- |
| | YP7 | 382 (2014) |
| GPRC5D | antibody FAB6300A | R&D Systems |
| GPRC5D | Antibody LS-A4180 | Lifespan Biosciences |
| HER2 | Antibody or fragment thereof, | U.S. Pat. No. 8,591,897 B2, |
| | Trastuzumab, Pertuzumab | WO2001000245 |
| HMWMAA | mAb9.2.27 | Kmiecik et al., Oncoimmunology |
| | | 3(1): e27185-3 (2014) |
| HMWMAA | Antibody or fragment thereof | U.S. Pat. No. 6,528,481; |
| | | WO2010033866; U.S. Patent |
| | | Application Publication |
| | | 20140004124 |
| Human telomerase | Antibody cat no: LS-B95-100 | Lifespan Biosciences |
| reverse | | |
| transcriptase | | |
| IGF-1 | Antibody or fragment thereof | U.S. Pat. No. 8,344,112 B2; |
| | | EP2322550 A1; WO2006138315; |
| | | WO2006138315 |
| IL-11Rα | Antibody or fragment thereof | Abcam ® (cat# ab55262); Novus |
| | | Biologicals (cat# EPR5446) |
| IL-11Rα | Peptide | Huang et al., Cancer Res. 72(1): 271- |
| | | 281 (2012) |
| IL-13Rα2 | Antibody or fragment thereof | WO2008146911, WO2004087758, |
| | | several commercial catalog |
| | | antibodies, and WO2004087758 |
| Intestinal carboxyl | Antibody 4F12: cat no: LS- | Lifespan Biosciences |
| esterase | B6190-50 | |
| KIT (also known as | Antibody or fragment thereof | U.S. Pat. No. 7,915,391, U.S. |
| CD117) | | Patent Application Publication |
| | | 20120288506, and several |
| | | commercial catalog antibodies |
| LewisY | Hu3S193 Ab (scFvs) | Kelly et al., Cancer Biother. |
| | | Radiopharm. 23(4): 411-423 (2008) |
| Lewis Y | NC10 scFv | Dolezal et al., Protein Engineering |
| | | 16(1): 47-56 (2003) |
| LMP2 | Antibody or fragment thereof | U.S. Pat. No. 7,410,640; U.S. Patent |
| | | Application Publication |
| | | 20050129701 |
| MAD-CT-2 | Antibody or fragment thereof | Rodman et al., J. Exp. Med. |
| | | 1(167): 1228-46 (1988); U.S. Pat. No. |
| | | 7,635,753 |
| MAGE-A1 | Antibody or fragment thereof | Willemsen et al., J. Immunol. |
| | | 174(12): 7853-7858 (2005) |
| MelanA/MART1 | Antibody or fragment thereof | EP2514766 A2; U.S. Pat. No. |
| | | 7,749,719 |
| Mesothelin | Antibody or fragment thereof | Morello et al., Cancer Discov. |
| | | 6: 133-46 (2016), Liu et al., Proc. |
| | | Natl. Acad. Sci. USA |
| | | 119: e2202439119-9 (2022) |
| MUC1 | Antibody SAR566658 | Commercial sources |
| MUC1c | Antibody or fragment thereof | Kufe, Oncogene 32: 1073-1081 |
| | | (2013) |
| MUC1* (truncated, | Antibody or fragment thereof | U.S. Patent Application Publication |
| membrane-bound) | | 20200239594A1 |
| Mut hsp70-2 | Monoclonal: cat no: LS- | Lifespan Biosciences |
| | C133261-100 | |
| NCAM also known | antibody clone 2-2B: | EMD Millipore |
| as CD56 | MAB5324, Lorvotuzumab | |
| NY-BR-1 | Antibody or fragment thereof | Jager et al., Appl. |
| | | Immunohistochem. Mol. Morphol. |
| | | 15(1): 77-83 (2007) |
| o-acetyl-GD2 | antibody 8B6 | Commercial sources |
| PDGFR-β | ab32570 | Abcam ® |
| PDGFRα | mAb APA5^{a} mAb 16A1, | Invitrogen ™; Biolegend, U.S. Pat. No. |
| | Olaratumab | 8,128,929 B2 |
| PLAC1 | Antibody or fragment thereof | Ghods et al., Biotechnol. Appl. |
| | | Biochem. 61(3): 363-369 (2013) |
| Polysialic acid | Antibody or fragment thereof | Nagae et al., J. Biol. Chem. |
| | | 288(47): 33784-33796 (2013) |
| PRSS21 | Antibody or fragment therof | U.S. Pat. No. 8,080,650 |
| PSCA | scFv 7F5 | Morgenroth et al., Prostate |
| | | 67(10): 1121-1131 (2007) |
| PSCA | scFv C5-II | Nejatollahi et al., J. of Oncology |
| | | article ID 839831-8 (2013) |
| PSCA | Antibody or fragment thereof | U.S. Patent Application Publication |
| | | 20090311181 |
| PSMA | Antibody or fragment thereof | Parker et al., Protein Expr. Purif. |
| | | 89(2): 136-145 (2013) |
| PSMA | J591 ScFv | U.S. Patent Application Publication |
| | | 20110268656 |
| PSMA | scFvD2B | Frigerio et al., European J. Cancer |
| | | 49(9): 2223-2232 (2013) |
| PSMA | mAbs 3/A12, 3/E7 and 3/F11 | WO2006125481 |
| | and single chain antibody | |
| | fragments scFv A5 and D7 | |
| RAGE-1 | Antibody MAB5328 | EMD Millipore |
| ROR1 | Antibody or fragment thereof | Hudecek et al., Clin. Cancer Res. |
| | | 19(12): 3153-3164 (2013); |
| | | WO2011159847; and U.S. Patent |
| | | Application Publication |
| | | 20130101607 |
| Sarcoma | Antibody or fragment thereof | Luo et al., EMBO Mol. Med. |
| translocation | | 4(6): 453-461 (2012) |
| breakpoints | | |
| sLe | antibody G193 (for lewis Y) | Scott et al., Cancer Res. 60: 3254- |
| | | 3261 (2000); Neeson et al., J. |
| | | Immunol. 190(1 Supplement): |
| | | 177.10 (2013) |
| Sperm protein 17 | Antibody or fragment thereof | Song et al., Target Oncol. 9(3): 263- |
| | | 272 (2013); Song et al., Med. Oncol. |
| | | 29(4): 2923-2931 (2012) |
| SSEA-4 | Antibody MC813 | Cell Signaling; other commercially |
| | | available antibodies |
| TAG72 | Antibody or fragment thereof | Hombach et al., Gastroenterology |
| | | 113(4): 1163-1170 (1997) |
| TAG72 | Ab691 | Abcam ® |
| TEM1/CD248 | Antibody or fragment thereof | Marty et al., Cancer Lett. |
| | | 235(2): 298-308 (2006); Zhao et al., |
| | | J. Immunol. Methods 363(2): 221- |
| | | 232 (2011) |
| Tie2 | Antibody AB33 | Cell Signaling Technology |
| Tn antigen | Antibody or fragment thereof | U.S. Pat. No. 8,440,798, Brooks et al., |
| | | PNAS 107(22): 10056-10061 (2010), |
| | | and Stone et al., OncoImmunology |
| | | 1(6): 863-873 (2012) |
| TNFRSF13B | Antibody or fragment thereof | WO2020247618A1 |
| (TACI) | | |
| TRP-2 | Antibody or fragment thereof | Wang et al., J. Exp. Med. |
| | | 184(6): 2207-2216 (1996) |
| TSHR | Antibody or fragment thereof | U.S. Patent 8,603,466; U.S. Pat. No. |
| | | 8,501,415; U.S. Pat. No. 8,309,693 |
| Tyrosinase | Antibody or fragment thereof | U.S. Pat. No. 5,843,674; U.S. Pat. No. |
| | | 5,843,674 |
| VEGFR2 | Antibody or fragment therof | Chinnasamy et al., J. Clin. Invest. |
| | | 120(11): 3953-3968 (2010) |
| WT-1 | Antibody 176ra33 | Dao et al., Sci. Transl. Med. |
| | | 5(176): 176ra33-22 (2013) |
| WT-1 | Antibody or fragment thereof | WO2012135854 |

In some embodiments that entail treatment of brain cancer, for example, the targeting ligand binds a brain tumor associated antigen. For example, tumor associated antigens present on GBM cells include ACVR1, EGFRVIII, IL13Rα2 and HER2. For example, the multiplexing approach may be used to treat brain cancer that simultaneously targets EGFRvIII, IL13Rα2and HER2. Other proteins that have been implicated in brain cancers and which may be targeted by the bifunctional compounds of the present disclosure include EphA2, CSPG4, GD2, PDGFRα and GRP78. Antibodies and/or functional fragments thereof that bind brain tumor associated antigens are known in the art. See, e.g., Table 1, above, which inter alia, describes antibodies and/or fragments thereof that bind ACVR1, PDGFRα, GD2 and EphA2. Targeting moieties that bind PDGFRα may include scFvs based on Olaratumab (and binding variants thereof).

In some embodiments, the targeting ligand binds to HER2 on HER2⁺ malignancies such as breast, lung, colorectal, brain, ovarian, and pancreatic cancer. Representative targeting ligands that bind HER and which may be useful in the present disclosure include Trastuzumab and Pertuzumab which bind the extracellular domains IV and II, respectively, of HER, and their HER-binding fragments (e.g., scFvs).

An antibody fragment that binds EGFRVIII, is described in O'Rourke, et al., Sci. Transl. Med. 9(399): eaaa0984-30 (2017). Other antibodies or fragments thereof that bind EGFRvIII are commercially available siltuximab and mAb DH8.3 (Novus Biologicals). Further representative examples of amino acid or gene sequences that encode scFvs targeting EGFRvIII that might be useful in the present disclosure are found in U.S. Patent Application Publication 2015/0259423.

An antibody fragment that binds IL13Rα2 is described in Brown, et al., N. Engl. J. Med. 375(26): 2561-2569 (2016). Other antibodies or fragments thereof that bind IL13Rα2 are commercially available from Abnova and Millipore.

An antibody fragment that binds HER2 is described in Ahmed, et al., JAMA Oncol. 3(8): 1049-1101 (2017). Other antibodies or fragments thereof that bind HER2 are commercially available, including trastuzumab and FRP5. Further representative examples of amino acid or gene sequences that encode scFvs targeting HER2 that might be useful in the present disclosure are found in U.S. Patent Application Publication 2011/0313137.

Another example of an antibody fragment that binds EphA2 is described in Chow, et al., Mol. Ther. 21 (3): 629-637 (2013). Yet other antibodies or fragments thereof that bind EphA2 are commercially available from Thermo Fisher (mAb4H5 and mAb 1C11A12) and RND Systems. Further representative examples of amino acid or gene sequences that encode for scFvs targeting EphA2 that might be useful in the present disclosure are described in U.S. Patent Application Publication 2010/436783.

An antibody fragment that binds CSPG4 is described in Pellegatta, et al., Sci. Transl. Med., 10: eaao2731-33 (2018). Another antibody that binds CSPG4 is described in Fenton et al., Oncol. Res. 22 (2): 117-21 (2015). Other antibodies or fragments thereof that bind CSPG4are commercially available bevacizumab and Creative Biolabs mAb 225.28. Yet other antibodies or fragments thereof that bind CSPG4 are commercially available from Aviva Systems Biology. Further representative examples of amino acid or gene sequences that encode scFvs targeting CSPG4 that might be useful in the present disclosure are described in U.S. Pat. No. 9,801,928 and U.S. Patent Application Publication 2019/0008940.

Another example of an antibody fragment that binds GD2 is described in Mount et al., Nat. Med. 24:572-579 (2018). Other antibodies or fragments thereof that bind GD2 include Dinutuximab, mAb 3F8, mAb 14g2a, and mAb 14.18. Further representative examples of amino acid or gene sequences that encode scFvs targeting GD2 that might be useful in the present disclosure are described in U.S. Pat. No. 4,675,287.

Another example of an antibody fragment that binds PDGFRα is described in Brennan et al., PLoS One, 4 (11): e7752-10 (2009). Other antibodies or fragments thereof that bind PDGFRα are commercially available from Abcam, LifeSpan Bio, Santa Cruz (sc-338) and Thermo Fisher (mAb APA5). Further representative examples of amino acid or gene sequences that encode scFvs targeting PDGFRα that might be useful in the present disclosure are described in U.S. Patent Application Publication 2012/0027767.

An antibody fragment that binds GRP78 is described in Kang et al., Sci. Rep. 6:34922-7 (2016). Other antibodies or fragments thereof that bind GRP78 are commercially available from Thermo Fisher (PA1-014A) and Abcam (N-20). Further representative examples of amino acid or gene sequences that encode scFvs targeting GRP78 that might be useful in the present disclosure are described in U.S. Pat. No. 10,259,884.

Other proteins that have been implicated in brain cancers and which may be targeted by the bifunctional compounds of the present disclosure include neural cell adhesion molecule (NCAM), cluster of differentiation 276 (CD276), and neuroectodermal stem cell marker (Nestin).

An antibody that binds NCAM is described in Modak et al., Cancer Res. 61:4048-4054 (2001). Other antibodies or fragments thereof that bind NCAM are mAb UJ13A and mAb ERIC-1. Further representative examples of amino acid or gene sequences that encode scFvs targeting NCAM that might be useful in the present disclosure are described in U.S. Pat. No. 7,402,560.

Antibodies or fragments thereof that bind Nestin are commercially available from Abcam (ab6142) and Novus Biologicals (NB100-1604). Antibodies or fragments thereof that bind βII-Tubulin are available from Abcam (2G10) and RND Systems (mAB 1195).

Another example of an antibody fragment that binds CS-1 is described in Chu et al., Blood, 122:14 (2013). Other antibodies or fragments thereof that bind CS-1 are commercially available and include REA150 (Miltenyi) or 162.1 (Biolegend). Further representative examples of amino acid or gene sequences that encode scFvs targeting CS-1 that might be useful in the present disclosure are described in International Publication Number WO 2004/100898 A2.

Another example of an antibody fragment that binds BCMA is described in Raje et al., N. Engl. J. Med. 380 (18): 1726-1737 (2019). Other antibodies or fragments thereof that bind BCMA are commercially available and include REA315 (Miltenyi), J6MO (Abeomics) (the anti-BCMA antibody included in the Belantamab conjugation compsosion), and 19F2(Biolegend). Further representative examples of amino acid or gene sequences that encode scFvs targeting BCMA that might be useful in the present disclosure are described in International Publication Numbers WO 2010/104949 A2 and WO 2003/014294 A2.

In some embodiments, the cell surface antigen is CD19, B-cell maturation antigen (BCMA), human epidermal growth factor receptor 2 (HER2), epidermal growth factor receptor (EGFR), CD38, CSI (SLAM family member 7 (SLAMF7)), G Protein-Coupled Receptor Class C Group 5 Member D (GPRC5D), or TNFRSF13B (TACI).

The transmembrane (TM) domain allows the CAR to be stably anchored into the cell membrane of the immune cell. The transmembrane domain may be derived from the same protein or from a different protein from which the other domains of the CAR are derived. The transmembrane domain may be derived from a natural or from a recombinant source. Where the source is natural, the domain may be derived from any membrane-bound or transmembrane protein. Representative examples of transmembrane domains that may be useful in the present disclosure include the transmembrane regions of CD28, CD27, CD3 epsilon, CD45, CD4,CD5, CD8, CD9, CD16, CD22, CD33, CD37, CD64, CD80, CD86, CD134, CD137, CD154.

The CAR can be designed to include a transmembrane domain that is indirectly attached to the extracellular binding domain. In such embodiments, the transmembrane domain is attached to the extracellular region of the CAR via a hinge domain. As used herein, the term “hinge domain” refers to a domain that links the extracellular binding domain to the transmembrane domain and may confer flexibility to the extracellular binding domain. In some embodiments, the hinge domain positions the extracellular domain close to the plasma membrane of the immune cell to minimize the potential for recognition by antibodies or binding fragments thereof. The hinge domain may be natural (such as a hinge from a human protein) or synthetic. Sources of hinge domains include human Ig (immunoglobulin) hinges (e.g., an IgG4 hinge, an IgD hinge), and a CD8 (e.g., CD8α hinge).

In some embodiments, the hinge domain is derived from cluster of differentiation 8 (CD8), for example SEQ ID NO: 50.

The intracellular signaling domain aids in immune cell activation upon binding of the CAR (e.g., 2nd generation, 3rd generation, engineered T cell receptor (TCR)) to the cell associated antigen on the target cell. Such domains are known in the art and are commonly referred to as second, third and fourth generation CARs and engineered T cell receptors (TCRs). An intracellular signaling domain is generally responsible for activation of at least one of the normal effector functions of the immune cell in which the CAR has been introduced. Examples of intracellular signaling domains include the cytoplasmic sequences of the TCR and co-receptors that act in concert to initiate signal transduction following antigen receptor engagement. As is known in the art, signals generated through the TCR alone are insufficient for full activation of T cells: therefore, a secondary or costimulatory signal is also required. Thus, T cell activation is mediated by two distinct classes of cytoplasmic signaling sequences, namely those that initiate antigen-dependent primary activation through the TCR (i.e., the primary intracellular signaling domains) and those that act in an antigen-independent manner to provide a secondary or costimulatory signal (i.e., the secondary cytoplasmic or costimulatory domain). The primary signaling domain regulates primary activation of the TCR complex either in a stimulatory way, or in an inhibitory way. Primary intracellular signaling domains that act in a stimulatory manner may contain signaling motifs known as immunoreceptor tyrosine-based activation motifs (ITAMs). Representative examples of ITAM-containing primary intracellular signaling domains that may be suitable for use in the present disclosure include those of CD3ζ, common FcRγ (FCER1G), Fc-γ RIIa, FcR-β (Fc-εR1b), CD3γ, CD3δ,and CD3ε. In some embodiments, the CARs include an intracellular signaling domain that contains the primary signaling domain of CD3ζ.

The intracellular signaling domain of the CAR may also include at least one other intracellular signaling or co-stimulatory domain. A co-stimulatory molecule is a cell surface molecule other than an antigen receptor or its ligands that is required for an efficient response of lymphocytes to an antigen. Representative examples of co-stimulatory domains that may be useful in the CARs of the present disclosure include CD27, CD28, 4-1BB (CD137), OX40, CD30, CD40, PD-1, ICOS, HVEM (LIGHTR), lymphocyte function-associated antigen-1 (LFA-1), CD2, CD7, LIGHT, NKG2C, and B7-H3. CD27 co-stimulation, for example, has been demonstrated to enhance expansion, effector function, and survival of human CAR T cells in vitro and augments human T cell persistence and antitumor activity in vivo (Song, et al., Blood 119 (3): 696-706 (2012)).

The intracellular signaling domain may be designed to include one or more, e.g., 1, 2, 3, 4, 5, or more costimulatory signaling domains, which may be linked to each other in a specified or random order, optionally via a linker molecule. Polypeptide linkers that are about 1-10 amino acids in length may join consecutive intracellular signaling sequences. Examples of such linkers include doublets such as Gly-Ser, and single amino acids, e.g., Ala and Gly. Combinations that may constitute the T-cell activation domain may be based on the cytoplasmic regions of CD28, CD137 (4-1BB), OX40 and HVEM, which serve to enhance T cell survival and proliferation: and CD3 CD3ζ and FcRε, which induce T cell activation. For example, CD3ζ, which contains 3 ITAMs, is the most commonly used intracellular domain component of CARS, transmits an activation signal to the T cell after antigen is bound. However, to provide additional co-stimulatory signaling, CD28 and OX40 domains can be used with CD3ζwhich enable the CAR—immune cells to transmit the proliferative/survival signals.

CARs that may be used in the present disclosure and methods of making them are known in the art and are described, for example, in U.S. Patent Application Publication 2018/0169109, each of which is incorporated herein by reference in its entirety. Additional CARs that may be useful have been approved by the FDA, and include tisagenlecleucel (Kymriah™), axicabtagene ciloleucel (Yescarta™), idecabtagene vicleucel (Abecma®), and lisocabtagene maraleucel (Breyanzi®), brexucabtagene autoleucel (Tecartus), and ciltacabtagene autoleucel (Carvykti).

Exemplary cytokine receptor switches and CAR constructs are shown below in Table 3.

TABLE 3
Cytokine receptor switch and CAR constructs.

| | CAR | | | | | | Cytokine Receptor Switch | | | | |
|---|---|---|---|---|---|---|---|---|---|---|---|
| scFv | FL | MPOB | AQ | DOTA | His | Others* | FL | MPOB | AQ | DOTA | His |

| | | | | | | | | | |
|---|---|---|---|---|---|---|---|---|---|
| 1 | CAR | | | | | | | | |
| 2 | | CAR | | | | | | | |
| 3 | | | CAR | | | | | | |
| 4 | | | | CAR | | | | | |
| 5 | | | | | CAR | | | | |
| 6 | | | | | | CAR | | | |
| 7 | | | | | | | IL2RA | | |
| 8 | | | | | | | IL2RB | | |
| 9 | | | | | | | IL2RG | | |
| 10 | | | | | | | IL4RA | | |
| 11 | | | | | | | IL7RA | | |
| 12 | | | | | | | IL9R | | |
| 13 | | | | | | | IL15RA | | |
| 14 | | | | | | | IL21R | | |
| 15 | CAR | | | | | | IL2RA | | |
| 16 | CAR | | | | | | IL2RB | | |
| 17 | CAR | | | | | | IL2RG | | |
| 18 | CAR | | | | | | IL4RA | | |
| 19 | CAR | | | | | | IL7RA | | |
| 20 | CAR | | | | | | IL9R | | |
| 21 | CAR | | | | | | IL15RA | | |
| 22 | CAR | | | | | | IL21R | | |
| 23 | | CAR | | | | | IL2RA | | |
| 24 | | CAR | | | | | IL2RB | | |
| 25 | | CAR | | | | | IL2RG | | |
| 26 | | CAR | | | | | IL4RA | | |
| 27 | | CAR | | | | | IL7RA | | |
| 28 | | CAR | | | | | IL9R | | |
| 29 | | CAR | | | | | IL15RA | | |
| 30 | | CAR | | | | | IL21R | | |
| 31 | | | | | | CAR | IL2RA | | |
| 32 | | | | | | CAR | IL2RB | | |
| 33 | | | | | | CAR | IL2RG | | |
| 34 | | | | | | CAR | IL4RA | | |
| 35 | | | | | | CAR | IL7RA | | |
| 36 | | | | | | CAR | IL9R | | |
| 37 | | | | | | CAR | IL15RA | | |
| 38 | | | | | | CAR | IL21R | | |
| 39 | | | | | | | IL2RA | | |
| | | | | | | | IL2RB | | |
| 40 | | | | | | | IL2RA | | |
| | | | | | | | IL2RB | | |
| | | | | | | | IL2RG | | |
| 41 | | | | | | | IL2RB | | |
| | | | | | | | IL2RG | | |
| 42 | | | | | | | IL4R | | |
| | | | | | | | IL2RG | | |
| 43 | | | | | | | IL7R | | |
| | | | | | | | IL2RG | | |
| 44 | | | | | | | IL9R | | |
| | | | | | | | IL2RG | | |
| 45 | | | | | | | IL15RA | | |
| | | | | | | | IL2RB | | |
| | | | | | | | IL2RG | | |
| 46 | | | | | | | IL21R | | |
| | | | | | | | IL2RG | | |
| 47 | | | | | | | IL2RB | IL2RA | |
| | | | | | | | IL2RG | | |
| 48 | | | | | | | IL2RG | IL4RA | |
| 49 | | | | | | | IL2RG | IL7RA | |
| 50 | | | | | | | IL2RG | IL9R | |
| 51 | | | | | | | IL2RB | IL15RA | |
| | | | | | | | IL2RG | | |
| 52 | | | | | | | IL2RG | IL21R | |
| 53 | | | | | | | IL2RG | IL2RA | IL2RB |
| 54 | | | | | | | IL2RG | IL4RA | |
| 55 | | | | | | | IL2RG | IL7RA | |
| 56 | | | | | | | IL2RG | IL9R | |
| 57 | | | | | | | IL2RG | IL15RA | IL2RB |
| 58 | | | | | | | IL2RG | IL21R | |
| 59 | | | | | | CAR | IL2RG | IL2RA | IL2RB |
| 60 | | | | | | CAR | IL2RG | IL4RA | |
| 61 | | | | | | CAR | IL2RG | IL7RA | |
| 62 | | | | | | CAR | IL2RG | IL9R | |
| 63 | | | | | | CAR | IL2RG | IL15RA | IL2RB |
| 64 | | | | | | CAR | IL2RG | IL21R | |

*CARs including CD19−, BCMA−, HER2−, and EGFR−CAR.

Table 3 illustrates exemplary combinations of CARs and cytokine receptor switches with same or different scFvs, that may be present in the immune cell.

### BAT-CARs

In some embodiments, the immune cells are binary activated T cells comprising nucleic acids encoding chimeric antigen receptors (BAT-CARs). BAT-CARs are substantially identical to CARs in terms of design, and the respective spacer domains (e.g., transmembrane, and intracellular domains). However, in contrast to CAR T cells that are engineered so directly bind a cell associated antigen such as a tumor associated antigen, BAT-CAR cells bind a synthetic antigen (which may be a masked pro-antigen or unmasked) that is not present on normal or cancer cells. The synthetic antigen is delivered and attached to a cell associated antigen in the form of a conjugate with an antibody or fragment thereof that binds the cell associated antigen. The BAT-CAR cells can be administered to a subject with a single conjugate or multiple conjugates that contain antibodies or fragments that bind different cell associated antigens. By uncoupling tumor cell targeting from tumor cell killing, a CAR T cell with a single specificity (to the synthetic antigen or unmasked pro-antigen) can simultaneously target a plurality of tumor associated antigens. Therefore, the design of BAT-CAR immune cells differs from CAR-immune cells mainly if not exclusively with respect to the extracellular binding domains.

The extracellular domain of a BAT-CAR is typically present at the amino terminal end and displayed on the surface of the immune cell. Except for its specificity, the extracellular domain of a BAT-CAR is typically an antibody or an antigen-binding fragment thereof such as an scFv.

Representative examples of synthetic antigens include fluorescein and fluorescein derivatives such as FITC. Representative examples of extracellular binding domains that bind fluorescein and FITC are described above (in connection with the cytokine receptor switches, per se). Representative examples of other binding moieties that may be useful as extracellular binding domains in a BAT-CAR are known in the art, e.g., 4M5.3 ScFv, disclosed in Midelfort et al. J. Mol. Biol. 343:685-701 (2004) and 2D12.5, 2D12.5ds, or C8.2.5, disclosed in Orcutt et al. Nucl. Med. Biol. 38 (2): 223-233 (2011). Representative examples of masked pro-antigens are known in the art, e.g., International Publication Nos WO2017/143094, WO2018/200713, WO2019/236522, and WO2020/006312, each of which is incorporated herein by reference.

A representative example of a masked pro-antigen and BAT-CAR is a BAT-CAR with specificity for fluorescein and a masked fluorescein pro-antigen. A stimulus, e.g., UV light unmasks the masked fluorescein molecule and thereby activates cells expressing the fluorescein-specific BAT-CAR. See. e.g., Kobayashi et al., ChemMedChem 17: e202100722-5 (2022), incorporated by reference. In some embodiments, the synthetic antigen is masked by the addition of one or more 5-carboxymethoxy-2-nitrobenzyl (CMNB) caging groups.

BAT-CARs that may be used in the present disclosure and methods of making them are known in the art and described, for example, in International Publication Nos WO2017/143094, WO2018/200713, WO2019/236522, and WO2020/006312, each of which is incorporated herein by reference in its entirety.

A representative example of a polynucleotide that encodes the BAT-CAR an anti-FL CAR-CD28-4-1BB-CD3ζ has the sequence designated as SEQ ID NO: 60:

| | |
|---|---|
| 1 | atggctctgc ctgtgacagc tctgctgctg cctctggctc tgcttctgca tgccgccaga |
| 61 | cctgacgtgg tcatgacaca gacacctctg agcctgcctg tgtctctggg agatcaggcc |
| 121 | agcatcagct gcagatctag ccagagcctg gtgcacagca acggcaacac ctacctgcgg |
| 181 | tggtatctgc agaagcccgg ccagtctcct aaggtgctga tctacaaggt gtccaacaga |
| 241 | gtgtccggcg tycccgatag attttctggc agcggctctg gcaccgactt caccctgaag |
| 301 | atcaatagag tggaagccga ggacctgggc gtgtacttct gtagccagtc tacccacgtg |
| 361 | ccatggacct ttggcggcgg aacaaagctg gaaatcaaga gcagcgccga cgacgccaag |
| 421 | aaggacgccg ctaagaagga tgacgccaaa aaagacgatg ccaaaaagga tggcggcgtg |
| 481 | aagctggacg aaacaggcgg aggacttgtt cagcctggcg gagccatgaa gctgagctgt |
| 541 | gtgaccagcg gcttcacctt cggccactac tggatgaact gggtccgaca gagccctgag |
| 601 | aaaggcctgg aatgggtcgc ccagttcaga aacaagccct acaactacga aacctactac |
| 661 | agcgacagcg tgaagggcag attcaccatc agccgggacg acagcaagtc cagcgtgtac |
| 721 | ctgcagatga acaacctgcg cgtggaagat accggcatct actactgtac cggcgccagc |
| 781 | tacggcatgg aatatctcgg ccagggcacc agcgtgaccg tgtctacaac aacccctgct |
| 841 | cctcggcctc ctacaccagc tcctacaatt gccagccagc cactgtctct gaggcccgaa |
| 901 | gcttgtagac ctgctgcagg cggagccgtg catacaagag gactggattt cgcctgcgac |
| 961 | ttctgggtgc tcgtggttgt tggcggagtg ctggcttgtt actccctgct ggttaccgtg |
| 1021 | gccttcatca tcttttgggt ccgaagcaag cggagccggc tgctgcacag cgactacatg |
| 1081 | aacatgaccc ctagacggcc cggacctacc agaaagcact accagcctta cgctcctcct |
| 1141 | agagacttcg ccgcctacag atccaagcgg ggcagaaaga agctgctgta catcttcaag |
| 1201 | cagcccttca tgcggcccgt gcagaccaca caagaggaag atggctgctc ctgcagattc |
| 1261 | cccgaggaag aagaaggcgg ctgcgagctg agagtgaagt tcagcagatc cgccgacgct |
| 1321 | cctgcctatc agcagggaca gaaccagctg tacaacgagc tgaacctggg gagaagagaa |
| 1381 | gagtacgacg tgctggacaa gcggagaggc agagatcctg agatgggcgg aaagccccag |
| 1441 | cggagaaaga atcctcaaga gggcctgtat aatgagctgc agaaagacaa gatggccgag |
| 1501 | gcctacagcg agatcggaat gaagggcgag cgcagaagag gcaagggaca cgatggactg |
| 1561 | taccagggcc tgagcaccgc caccaaggat acctatgatg ccctgcacat gcaggccctg |
| 1621 | ccacctagat gatga |

### Introduction of Polynucleotides Encoding Cytokine Receptor Switches into Immune Cells

Immune cells such as T cells may be engineered to comprising nucleic acids encoding express cytokine receptor switches in accordance with known techniques. Generally, a polynucleotide vector is constructed that encodes the cytokine receptor switch and the vector is introduced (e.g., transfected, or transduced) into a population of immune cells. The cells are then grown under conditions promoting expression of the polynucleotide encoding the cytokine receptor switch. Successful transfection (or transduction which refers to viral-mediated gene integration) and display of cytokine receptor switches may be conducted via standard techniques. In some embodiments, immune cells may be engineered to produce cytokine receptor switches by first constructing a retroviral vector encoding a selected cytokine receptor switch. Retroviral transduction may be performed using known techniques (e.g., Johnson, et al., Blood 114:535-546 (2009)). The surface expression of cytokine receptor switch on transfected immune cells may be determined, for example, by flow cytometry.

Expression vectors that encode the cytokine receptor switches can be introduced as one or more DNA molecules or constructs, where there may be at least one marker that will allow for selection of host cells that contain the construct(s).

A DNA construct is an artificially constructed segment of nucleic acid for the introduction (i.e., transfection or transduction) into a target cell or tissue. The term “nucleic acid” as used herein refers to a polymer of nucleotides, each of which are organic molecules consisting of a nucleoside (a nucleobase and a five-carbon sugar) and a phosphate. The term nucleotide, unless specifically sated or obvious from context, includes nucleosides that have a ribose sugar (i.e., a ribonucleotide that forms ribonucleic acid, RNA) or a 2′-deoxyribose sugar (i.e., a deoxyribonucleotide that forms deoxyribonucleic acid, DNA). Nucleotides serve as the monomeric units of nucleic acid polymers or polynucleotides. The four nucleobases in DNA are guanine (G), adenine (A), cytosine (C) and thymine (T). The four nucleobases in RNA are guanine (G), adenine (A), cytosine (C) and uracil (U). Nucleic acids are linear chains of nucleotides (e.g., at least 3 nucleotides) chemically bonded by a series of ester linkages between the phosphoryl group of one nucleotide and the hydroxyl group of the sugar (i.e., ribose or 2′-deoxyribose) in the adjacent nucleotide.

The nucleic acids encode at least a cytokine receptor switch protein. The terms “protein” and “polypeptide” as used herein refer to a string of amino acids connected by amide linkages, typically at least ten (10) amino acids or longer in length. Proteins are ordinarily derived from organisms but are not limited thereto, and for example, they may be composed of an artificially designed sequence. They may also be any of naturally derived proteins, synthetic proteins, or recombinant proteins.

The constructs can be prepared in conventional ways, where the individual components of the cytokine receptor switches may be ligated in the desired order, cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagenesis, etc., as appropriate. The construct(s) once completed and demonstrated to have the appropriate sequences may then be packaged into a suitable vector which is then introduced into the immune cell (i.e., T cell) by any convenient means. Vectors containing useful elements such as bacterial or yeast origins of replication, selectable and/or amplifiable markers (e.g., hypoxanthine-guanine phosphoribosyltransferase (hprt), neomycin resistance, thymidine kinase, hygromycin resistance, etc.), promoter/enhancer elements for expression in prokaryotes or eukaryotes, one or more suitable sites for the insertion of the nucleic acid sequences, such as a multiple cloning site (MCS), and etc. that may be used to prepare stocks of construct DNAs and for carrying out transfections are well known in the art, and many are commercially available.

The constructs may be integrated and packaged into non-replicating, defective viral genomes like Adenovirus, Adeno-associated virus (AAV), or Herpes simplex virus (HSV) or others, including retroviral vectors or lentiviral vectors, for infection or transduction into cells. The constructs may include viral sequences for transfection, if desired. Alternatively, the construct may be introduced by fusion, electroporation, biolistics, transfection, lipofection, or the like. The host immune cells may be grown and expanded in culture before introduction of the construct(s), followed by the appropriate treatment for introduction of the construct(s) and integration of the construct(s). The cells are then expanded and screened by virtue of a marker present in the construct.

In some instances, the construct may be engineered to have a target site for homologous recombination, where it is desired that the construct be integrated at a particular genomic locus. For example, an endogenous gene can be knocked out and replaced (at the same locus or elsewhere) with the gene(s) encoded for by the construct using materials and methods as are known in the art for homologous recombination. For homologous recombination, one may use either OMEGA or O-vectors. See, e.g., Thomas and Capecchi, Cell 51:503-512 (1987): Mansour et al., Nature 336:348-352 (1988); and Joyner et al., Nature 338:153-156 (1989).

In some embodiments, the vector is a a lentiviral vector or a recombinant lentivirus vector. In some embodiments, the expression vector is a non-integrative and non-replicative recombinant lentivirus vector. Exemplary lentiviral vectors include, for example, LentiVector and LentiStable from Oxford BioMedica, LV-Max from Gibco, and the like. The construction of lentiviral vectors has been described, for example, in U.S. Pat. No. 5,665,577, 5,981,276, 6,013,516, 7,090,837, 8,119,119 and 10,954,530.

### Formulations and Methods of Use

### Formulations Containing Cytokine Receptor Switch-Containing Immune Cells

The inventive immune cells may be formulated in pharmaceutically acceptable vehicles or carriers, the selection and amounts of which may be determined depending upon the mode of administration. The therapeutically effective amount of the formulation may depend upon the concentration of the cells in the overall volume of the formulation. The number of inventive immune cells administered to a subject may vary between wide limits, depending various factors including, for example, the location, type, and severity of the cancer, and the age and condition of the individual to be treated, and is within the level of skill of a treating physician. In general, formulations contain from about 1×10⁴ to about 1×10¹⁰ inventive immune cells. In some embodiments, the formulation contains from about 1×10⁵ to about 1×10⁹ inventive immune cells, from about 5×10⁵ to about 5×10⁸ inventive immune cells, or from about 1×10⁶ to about 1×10⁷ inventive immune cells. See, for example, International Publication No WO/2020/006312, which is incorporated herein by reference in its entirety.

The formulation of inventive immune cells may be administered to a subject in need thereof in accordance with acceptable medical practice. An exemplary mode of administration is intravenous injection. Other modes of administration may include intratumoral, intradermal, subcutaneous (s.c., s.q., sub-Q. Hypo), intramuscular (i.m.), intraperitoneal (i.p.), intra-arterial, intramedullary, intracardiac, intra-articular (joint), intrasynovial (joint fluid area), intracranial (including convection-enhanced delivery), intraspinal, and intrathecal (spinal fluids). Any known device useful for parenteral injection or infusion of the formulations can be used to affect such modes of administration. Representative vehicles and carriers include buffers such as neutral buffered saline, phosphate buffered saline and the like. The compositions may further include one or more pharmaceutically acceptable excipients. Examples of such excipients include carbohydrates such as glucose, mannose, sucrose or dextrans, mannitol: proteins: polypeptides or amino acids such as glycine: antioxidants: chelating agents such as EDTA or glutathione: adjuvants (e.g., aluminum hydroxide): and preservatives.

### Formulations Containing the Synthetic Small Molecule

To stimulate the inventive immune cells in vivo, A therapeutically effective amount of the synthetic small molecule may be administered to a subject in need thereof in accordance with acceptable medical practice. The synthetic small molecule may be formulated in pharmaceutically acceptable vehicles or carriers, the selection and amounts of which may be determined depending upon the mode of administration. An exemplary mode of administration is intravenous injection. Other modes include intratumoral, intradermal, subcutaneous (s.c., s.q., sub-Q, Hypo), i.m., i.p., intra-arterial, intramedullary, intracardiac, intra-articular, intrasynovial, intracranial, intraspinal, and intrathecal. Any known device useful for parenteral injection or infusion of the formulations can be used to effect administration of the synthetic small molecule. A representative example of a pharmaceutically acceptable vehicle or carrier is serum albumin such as human serum albumin, dextrans, and antibodies.

In general, the amount of the synthetic small molecule ranges from 0.1 to 1000 μg/mL based on total volume of the composition. In some embodiments, the amount of the synthetic small molecule ranges is about 0.1 to 100 μg/mL based on total volume of the composition. The total amount of the small molecule may differ depending on the vehicle or carrier. For example, for in vivo injection, the effective dose may be higher than ex vivo because not all the injected small molecules might be delivered to the tumor.

### Methods for Stimulating Immune Cells Expressing Inventive Cytokine Receptor Switches

Broadly, the inventive methods entail treating or contacting the immune cells with a sufficient concentration of the synthetic small molecule.

In some embodiments, the method is conducted ex vivo. Immune cells that contain an exogenous nucleic acid encoding the cytokine receptor switch are placed in a suitable container suitable medium and contacted with an effective amount of the synthetic small molecule, e.g., from 0.1 to 1000 μg/mL based on total volume of the medium. In some embodiments, the amount of the synthetic small molecule ranges is about 0.1 to 100 μg/mL based on total volume of the medium. Representative examples of suitable media that may be used in the practice of the methods include RPMI-1640 (Gibco™) and X-VIVO™ 15 (BioWhittaker™).

The duration of the contact (also referred to herein as “treatment” or “treating” or “stimulating”) may be in the order of hours, days and even weeks (e.g., 1, 2, 3, 4 or more weeks). In some embodiments, the contacting may be conducted in a high-affinity plate, dish, or flask wherein the small molecule is conjugated to the carrier affixed to a surface of the container. Representative examples of carriers include bovine or human serum albumin, dextran, and antibodies (e.g., anti-HER2 antibodies, anti-EGFR antibodies, anti-BCMA antibodies, and anti-CD19 antibodies). In some embodiments, the antibody is Pertuzumab, Cetuximab, Belentamab, J6M0, or Daratuzumab.

In some embodiments, stimulation promotes proliferation or a change in phenotype of the immune cells. For example, the stimulation may promote an increase in the population of the stimulated immunes cells by at least 10%, 20%, 30%, 40%, 50%, 60%, 70%, 80%, 90%, 95%, 100%, or greater.

In some embodiments, the immune cell stimulation promotes a change in phenotype of the immune cells. Exemplary phenotypes include memory, cytotoxic, and regulatory phenotypes.

In some embodiments, the immune cells comprising a nucleic acid encoding a cytokine receptor switch are stimulated on fluorescein isothiocyanate (FITC)-conjugated-bovine serum albumin (BSA)-coated plate with increasing doses of FITC (0, 0.1, 1, 10, 100, and 1000 μg/mL) in the presence of CD3/CD28 co-stimulation for up to 2 weeks. Central memory (CD45RA−, C-C chemokine receptor type 7 (CCR7)⁺) and effector memory (CD45RA⁻ CCR7⁻) markers on CD4⁺ and CD8⁺ T cells may be assessed by flow cytometry.

In some embodiments, immune cells comprising a nucleic acid encoding a cytokine receptor switch are stimulated on FITC-conjugated-BSA-coated plate with increasing doses of FITC (0, 0.1, 1, 10, 100, and 1000 μg/mL) in the presence or absence of CD3/CD28 co-stimulation for up to 2 weeks. Effector memory (CD45RA CCR7), central memory (CD45RA−, CCR7⁺), and activation (CD69) markers on CD4⁺ and CD8⁺ T cells may be assessed by flow cytometry.

In some embodiments, the immune cells comprising a nucleic acid encoding a cytokine receptor switch are stimulated on carboxyfluorescein-conjugated-BSA-coated plate with increasing doses of carboxyfluorescein (0, 0.1, 1, 10, 100, and 1000 μg/mL) in the presence of CD3/CD28 co-stimulation for up to 2 weeks.

In some embodiments, immune cells comprising a nucleic acid encoding a CAR with or without one or a combination of different cytokine receptor switches are stimulated on small molecule-conjugated-antibody-coated plates with increasing doses of small molecule conjugates (0, 0.1, 1, 10, and 100 μg/mL) in the presence or absence of CD3/CD28 co-stimulation. Expression of IL-2, IFN-gamma and CD69 in CD4⁺ and CD8⁺ T cells may be assessed by flow cytometry.

The stimulated immune cells may be isolated from the medium and then formulated for delivery to a subject. In some embodiments, the immune cells are stimulated in vivo.

These embodiments entail administering to a subject in need thereof a therapeutically effective amount of the composition described herein: and administering to the subject a therapeutically effective amount of the synthetic small molecule. The synthetic small molecule and the immune cells may be administered via the same or different formulations and substantially simultaneously or sequentially. The method may further include administering the subject a formulation of synthetic monomeric or polymeric small molecules that serves to decrease the stimulation.

Administration of the ex vivo activated immune cells and the activation of the immune cells in vivo are typically performed in the context of treating a disease or disorder, namely cancer.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present disclosure may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject is suffering from the disease or disorder. Thus, subjects suffering from, and suspected of suffering from, a specific disease or disorder are not necessarily two distinct groups.

Broadly, the methods may be effective in the treatment of carcinomas (solid tumors including both primary and metastatic tumors), sarcomas, melanomas, and hematological cancers (cancers affecting blood including lymphocytes, bone marrow and/or lymph nodes) such as leukemia, lymphoma, and multiple myeloma. Adult tumors/cancers and pediatric tumors/cancers are included. The cancers may be vascularized, or not yet substantially vascularized, or non-vascularized tumors.

Representative examples of cancers include adrenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., gliomas and glioblastomas such as brain stem glioma, gestational trophoblastic tumor glioma, cerebellar astrocytoma, cerebral astrocytoma/malignant glioma, ependymoma, medulloblastoma, supratentorial primitive neuroectodeimal tumors, visual pathway and hypothalamic glioma), breast cancer, bronchial adenomas/carcinoids, carcinoid tumor, nervous system cancer (e.g., central nervous system cancer, central nervous system lymphoma), cervical cancer, chronic myeloproliferative disorders, colorectal cancer (e.g., colon cancer, rectal cancer), lymphoid neoplasm, mycosis fungoids, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., stomach cancer, small intestine cancer, gastrointestinal carcinoid tumor, gastrointestinal stromal tumor (GIST)), cholangiocarcinoma, germ cell tumor, ovarian germ cell tumor, head and neck cancer, neuroendocrine tumors, Hodgkin's lymphoma, Ann Arbor stage III and stage IV childhood Non-Hodgkin's lymphoma, ROS1-positive refractory Non-Hodgkin's lymphoma, leukemia, lymphoma, multiple myeloma, hypopharyngeal cancer, intraocular melanoma, ocular cancer, islet cell tumors (endocrine pancreas), renal cancer (e.g., Wilm's Tumor, renal cell carcinoma), liver cancer, lung cancer (e.g., non-small cell lung cancer and small cell lung cancer), ALK-positive anaplastic large cell lymphoma, ALK-positive advanced malignant solid neoplasm, Waldenstrom's macroglobulinema, melanoma, intraocular (eye) melanoma, merkel cell carcinoma, mesothelioma, metastatic squamous neck cancer with occult primary, multiple endocrine neoplasia (MEN), myelodysplastic syndromes, myelodysplastic/myeloproliferative diseases, nasopharyngeal cancer, neuroblastoma, oral cancer (e.g., mouth cancer, lip cancer, oral cavity cancer, tongue cancer, oropharyngeal cancer, throat cancer, laryngeal cancer), ovarian cancer (e.g., ovarian epithelial cancer, ovarian germ cell tumor, ovarian low malignant potential tumor), pancreatic cancer, islet cell pancreatic cancer, paranasal sinus and nasal cavity cancer, parathyroid cancer, penile cancer, pharyngeal cancer, pheochromocytoma, pineoblastoma, metastatic anaplastic thyroid cancer, undifferentiated thyroid cancer, papillary thyroid cancer, pituitary tumor, plasma cell neoplasm/multiple myeloma, pleuropulmonary blastoma, prostate cancer, retinoblastoma, rhabdomyosarcoma, salivary gland cancer, uterine cancer (e.g., endometrial uterine cancer, uterine sarcoma, uterine corpus cancer), squamous cell carcinoma, testicular cancer, thymoma, thymic carcinoma, thyroid cancer, juvenile xanthogranuloma, transitional cell cancer of the renal pelvis and ureter and other urinary organs, urethral cancer, gestational trophoblastic tumor, vaginal cancer, vulvar cancer, hepatoblastoma, rhabdoid tumor, and Wilms tumor.

Sarcomas that may be treatable with the methods of the present disclosure include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue), mesenchymous or mixed mesodermal tumor (mixed connective tissue types), and histiocytic sarcoma (immune cancer).

In some embodiments, methods of the present disclosure entail treatment of subjects having cell proliferative diseases or disorders of the hematological system, liver, brain, lung, colon, pancreas, prostate, ovary, breast, skin, and endometrium.

As used herein, “cell proliferative diseases or disorders of the hematological system” include lymphoma, leukemia, myeloid neoplasms, mast cell neoplasms, myelodysplasia, benign monoclonal gammopathy, lymphomatoid papulosis, polycythemia vera, agnogenic myeloid metaplasia, and essential thrombocythemia. Representative examples of hematologic cancers may thus include multiple myeloma, lymphoma (including T-cell lymphoma. Hodgkin's lymphoma, non-Hodgkin's lymphoma (diffuse large B-cell lymphoma (DLBCL), follicular lymphoma (FL), mantle cell lymphoma (MCL) and ALK+ anaplastic large cell lymphoma (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, metastatic pancreatic adenocarcinoma, refractory B-cell non-Hodgkin's lymphoma, and relapsed B-cell non-Hodgkin's lymphoma, childhood lymphomas, and lymphomas of lymphocytic and cutaneous origin, e.g., small lymphocytic lymphoma, leukemia, including childhood leukemia, hairy-cell leukemia, acute lymphocytic leukemia, acute myelocytic leukemia, acute myeloid leukemia (e.g., acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms.

Multiple myeloma is a cell proliferative disease or disorder of the hematological system. Multiple myeloma is a plasma cell neoplasm. Healthy plasma cells produce antibodies that recognize and attack pathogens. In multiple myeloma, cancerous plasma cells accumulate in the bone marrow and crowd out healthy blood cells. Rather than produce antibodies, the cancerous myeloma plasma cells produce abnormal proteins that can cause complications. The overgrowth of plasma cells also results in crowding out of normal blood-forming cells, leading to low blood counts as well as thrombocytopenia and leukopenia. The myeloma plasma cells make abnormal antibodies known as monoclonal immunoglobulin, monoclonal protein (M-protein), M-spike, or paraprotein.

Other plasma cell cancers that do not meet the multiple myeloma criteria include monoclonal gammopathy of uncertain significance (MGUS), smoldering multiple myeloma (SMM), solitary plasmacytoma, and light chain amyloidosis. Minimal residual disease (MRD) refers to the small number of malignant cells below the limit of detection available with conventional morphologic assessment. MRD in multiple myeloma refers to myeloma cells that are present in the bone marrow after measuring a clinical response (CR) and the subject is in remission. These residual myeloma cells are clinically relevant, as they may lead to disease progression and relapse.

As used herein, “cell proliferative diseases or disorders of the liver” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the liver may include hyperplasia, metaplasia, and dysplasia of the liver.

As used herein, “cell proliferative diseases or disorders of the brain” include all forms of cell proliferative disorders affecting the brain. Cell proliferative disorders of the brain may include brain cancer (e.g., gliomas, glioblastomas, meningiomas, pituitary adenomas, vestibular schwannomas, and primitive neuroectodermal tumors (medulloblastomas)), a precancer or precancerous condition of the brain, benign growths or lesions of the brain, and malignant growths or lesions of the brain, and metastatic lesions in tissue and organs in the body other than the brain. Cell proliferative disorders of the brain are also called central nervous system (CNS) tumors, which include astrocytic tumors, oligodendroglial tumors, mixed gliomas, ependymal tumors, medulloblastomas, pineal parenchymal tumors, meningeal tumors, germ cell tumors, craniopharyngioma (grade I). Cell proliferative disorders of the brain may include hyperplasia, metaplasia, and dysplasia of the brain. A cancer that has spread to the brain is referred to as a metastatic brain tumor. Approximately half of metastic brain tumors originate from lung tumors. Other tumors that have a propensity to spread to the brain include, for example, melanoma, breast cancer, colon cancer, kidney cancer, and nasopharyngeal cancer.

Glioblastoma (GBM), also referred to as a grade IV astrocytoma, is a fast-growing and aggressive cell proliferative disorder of the brain. GBM is an astrocytic tumor that begins in astrocytes (a glial cell type) and may also be called gliomas. GBM invades the nearby brain tissue, but generally does not spread to distant organs. GBMs can arise in the brain de novo or evolve from a lower-grade astrocytoma.

As used herein, “cell proliferative diseases or disorders of the lung” include all forms of cell proliferative disorders affecting lung cells. Cell proliferative disorders of the lung include lung cancer, precancer and precancerous conditions of the lung, benign growths or lesions of the lung, hyperplasia, metaplasia, and dysplasia of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), adenocarcinoma, small cell carcinoma, large cell carcinoma, squamous cell carcinoma, and mesothelioma. Lung cancer can include “scar carcinoma”, bronchioveolar carcinoma, giant cell carcinoma, spindle cell carcinoma, and large cell neuroendocrine carcinoma. Lung cancer also includes lung neoplasms having histologic and ultrastructural heterogeneity (e.g., mixed cell types). In some embodiments, a compound of the present disclosure may be used to treat non-metastatic or metastatic lung cancer (e.g., NSCLC, ALK-positive NSCLC, NSCLC harboring ROSI rearrangement, lung adenocarcinoma, and squamous cell lung carcinoma).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer, malignant colon neoplasms, carcinoma in situ, typical carcinoid tumors, and atypical carcinoid tumors, adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polyposis, Gardner's syndrome, Peutz-Jeghers syndrome, Turcot's syndrome and juvenile polyposis. Cell proliferative disorders of the colon may also be characterized by hyperplasia, metaplasia, or dysplasia of the colon.

As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the ovary” include all forms of cell proliferative disorders affecting cells of the ovary. Cell proliferative disorders of the ovary involve growth of cells that forms in one or both ovaries, the fallopian tubes, or the tissue that covers organs in the abdomen (e.g., cystadenocarcinoma, ovarian embryonal carcinoma, ovarian adenocarcinoma) Cell proliferative disorders of the ovary may include a precancer or precancerous condition of the ovary, benign growths or lesions of the ovary, ovarian cancer, and metastatic lesions in tissue and organs in the body other than the ovary. Cell proliferative disorders of the ovary may include hyperplasia, metaplasia, and dysplasia of the ovary. Cell proliferative disorders of the ovary include ovarian epithelial cancer (epithelial ovarian carcinomas), germ cell tumors, and stromal cell tumors. Epithelial ovarian carcinomas are the most common type of ovarian cancer. About 85% to 90% of these cancers involve the cells that cover the outer surface of the ovary. They commonly spread first to the lining and organs of the pelvis and abdomen and then to other parts of the body. Nearly 70% of women with this type of ovarian cancer are diagnosed in the advanced stages. Ovarian epithelial cancer, fallopian tube cancer, and primary peritoneal cancer are epithelial ovarian carcinomas.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast. Cell proliferative disorders of the breast are a group of disorders in which cells in the breast grow out of control. Cell proliferative disorders of the breast may include hyperplasia, metaplasia, and dysplasia of the breast. Cell proliferative disorders of the breast can begin in different parts of the breast. A breast is made up of three main parts: lobules, ducts, and connective tissue. The lobules are the glands that produce milk. The ducts are tubes that carry milk to the nipple. The connective tissue (which consists of fibrous and fatty tissue) surrounds and connects the breast tissue. Most breast cancers begin in the ducts or lobules. Invasive ductal carcinoma and invasive lobular carcinoma are the two most common types of breast cancer. In invasive ductal carcinoma, cancer cells originate in the ducts and then spread, or metastasize, outside the ducts into other parts of the breast tissue. In invasive lobular carcinoma, cancer cells originate in the lobules and then spread from the lobules to the breast tissues that are close by. As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the skin.

As used herein, “cell proliferative diseases or disorders of the endometrium” include all forms of cell proliferative disorders affecting cells of the endometrium. Cell proliferative disorders of the endometrium may include a precancer or precancerous condition of the endometrium, benign growths or lesions of the endometrium, endometrial cancer, and metastatic lesions in tissue and organs in the body other than the endometrium. Cell proliferative disorders of the endometrium may include hyperplasia, metaplasia, and dysplasia of the endometrium.

In some embodiments, the cancer is breast cancer, ovarian cancer, multiple myeloma, lung cancer, or glioblastoma multiforme. The therapeutic methods of the present disclosure may be “first-line”, i.e., an initial treatment in patients who not yet undergone any anti-cancer treatment, either alone or in combination with other treatments. The therapeutic methods of the present disclosure may be advantageously used as a “second-line” therapy in that they are administered to patients who have undergone at least one prior anti-cancer treatment regimen, e.g., chemotherapy, radioimmunotherapy, toxin therapy, prodrug-activating enzyme therapy, antibody therapy, surgical therapy, immunotherapy, radiation therapy, targeted therapy or any combination thereof either alone or in combination with other treatments. In some cases, the prior therapy may have been unsuccessful or partially successful but where the patient became intolerant to the particular treatment, and particularly in cases where the front-line therapy is no longer effective on account of antigen loss/escape.

### Combination Therapy

In certain embodiments, the inventive methods of treating cancer may be part of a combination therapy wherein the subject is also treated with another agent that exerts an indirect or direct effect. In the case of administration of BAT-CAR immune cells,, the other agent is a synthetic antigen that is conjugated to a binding moiety that binds a cell surface antigen such as a tumor associated antigen. The BAT-CAR immune cells bind the cell surface antigen indirectly via the extracellular binding domain of the BAT-CAR which binds the synthetic antigen. Representative examples of synthetic antigens and conjugates thereof are described, for example, in International Publication Nos WO2017/143094, WO2018/200713,and WO2020/006312, each which is incorporated herein by reference in its entirety. In some embodiments, the synthetic antigen is masked or caged (a “pro-antigen”) which renders it unable to bind the extracellular binding domain of the BAT-CAR. These embodiments require administration of a further agent that unmasks or uncages the antigen so that it can interact with the extracellular binding domain of the BAT-CAR. This feature adds yet another level of control to the inventive methods. Representative examples of synthetic pro-antigens and conjugates thereof are described, for example, in International Publication Nos WO2017/143094, WO2018/200713, and WO2020/006312, which is incorporated herein by reference in its entirety. In some embodiments, the synthetic antigen is masked by the addition of one or more CMNB caging groups and the unmasking agent is UV light.

In some embodiments, the methods entail administration of another anti-cancer agent. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cancer cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo-and radiotherapy by combining it with other therapies. In the context of the present disclosure, it is contemplated that cell therapy could be used similarly in conjunction with chemotherapeutic, radiotherapeutic, or immunotherapeutic intervention, as well as pro-apoptotic or cell cycle regulating agents.

Alternatively, the present inventive therapy may precede or follow the other agent treatment by intervals ranging from minutes to weeks. In embodiments where the other agent and present disclosure are applied separately to the individual, one would generally ensure that a significant period of time did not expire between the times of each delivery, such that the agent and inventive therapy would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It is expected that the treatment cycles would be repeated as necessary. It also is contemplated that various standard therapies, as well as surgical intervention, may be applied in combination with the inventive cell therapy.

Multiple myeloma therapeutics that are suitable for the combination with the inventive therapies described herein include belantamab mafodotin-blmf (Blenrep®), bortezomib (Velcade®), carfilzomib (Kyprolis®), carmustine (BICNU®), ciltacabtagene autoleucel (Carvykti®), cyclophosphamide, daratumumab (Darzalex®), daratumumab and hyaluronidase-fihj (Darzalex Faspro®), doxorubicin hydrochloride liposome (Doxil®), elotuzumab (Empliciti®), idecabtagene vicleucel (Abecma®), isatuximab-irfc (Sarclisa®), ixazomib citrate (Ninlaro®), lenalidomide (Revlimid), melphalan and melphalan hydrochloride (Alkeran® Tablets, Alkeran® for injection, Evomela®), pamidronate disodium (Aredia®), plerixafor (Mozobil®), pomalidomide (Pomalyst®), Selinexor (Xpovio®), thalidomide (Thalomid®), zoledronic acid (Zometa®), and the PAD combination of bortezomib (PS-341), doxorubicin hydrochloride (Adriamycin®), and dexamethasone.

Breast cancer prevention and therapeutics that are suitable for the combination with the inventive therapies described herein may also include raloxifene and tamoxifen citrate (Soltamox®), abemaciclib (Verzenio®), paclitaxel (Abraxane®), ado-trastuzumab emtansine (Kadcyla®), everolimus (Afinitor®, Zortress®, Afinitor Disperz®), alpelisib (Piqray®), anastrozole (Arimidex®), pamidronate disodium (Aredia®), exemestane (Aromasin®), cyclophosphamide, doxorubicin hydrochloride, epirubicin hydrochloride (Ellence®), fam-trastuzumab deruxtecan-nxki (Enhertu®), fluorouracil (5-FU: Adrucil®), toremifene (Fareston®), letrozole (Femara®), gemcitabine (Gemzar®, Infugem®), eribulin mesylate (Halaven®), trastuzumab and hyaluronidase-oysk (Herceptin Hylecta®), trastuzumab (Herceptin®), palbociclib (Ibrance®), ixabepilone (Ixempra®), pembrolizumab (Keytruda®), ribociclib (Kisqali®), olaparib (Lynparza®), margetuximab-cmkb (Margenza®), neratinib maleate (Nerlynx®), pertuzumab (Perjeta®), pertuzumab trastuzumab and hyaluronidase-zzxf (Phesgo®), talazoparib tosylate (Talzenna®), docetaxel (Taxotere®), atezolizumab (Tecentriq®), thiotepa (Tepadina®), methotrexate sodium (Trexall®), sacituzumab govitecan-hziy (Trodelvy®), tucatinib (Tukysa®), lapatinib ditosylate (Tykerb®), vinblastine sulfate, capecitabine (Xeloda®), and goserelin acetate (Zoladex®).

Ovarian cancer therapeutics that are suitable for the combination with the inventive therapies described herein include melphalan (Alkeran®), bevacizumab (Alymsys®, Avastin®, Mvasi®, Zirabev®), cisplatin, cyclophosphamide, doxorubicin hydrochloride, doxorubicin hydrochloride liposomes (Doxil®), gemcitabine hydrochloride (Gemzar®, Infugem®), topotecan hydrochloride (Hycamtin®), olaparib (Lynparza®), carboplatin (Paraplatin®), rucaparib camsylate (Rubraca®), thiotepa (Tepadina®), and niraparib tosylate monohydrate (Zejula®). Drugs approved for treating epithelial ovarian carcinomas may be applied to ovarian germ cell cancers.

Brain cancer therapeutics that are suitable for the combination with the inventive therapies described herein include Belzutifan (Welireg), Bevacizumab (Alymsys, Avastin, Mvasi, Zirabev), Carmustine (BiCNU), Carmustine Implant (Gliadel Wafer), Everolimus (Afinitor, Afinitor Disperz), Lomustine, Naxitamab-gqgk (Danyelza), and Temozolomide (Temodar)

### Immunotherapy

Checkpoint inhibitors (e.g., pembrolizumab (Keytruda®), nivolumab (Opdivo®), cemiplimab (Libtayo®), atezolizumab (Tecentriq®), avelumab (Bavencio®), durvalumab (Imfinzi®) or other immune modulating anti-bodies or reagents may also be used as part of a combined therapy, in conjunction with the present cell therapy.

### Chemotherapy

Cancer therapies also include a variety of combination therapies with both chemical and radiation-based treatments. Combination chemotherapies include, for example, Abraxane®, altretamine, docetaxel, Herceptin®, methotrexate, Novantrone®, Zoladex®), cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, Taxol®, gemcitabien, Navelbine®, farnesyl-protein tansferase inhibitors, transplatinum, 5-fluorouracil, vincristine, vinblastine and methotrexate, or any analog or derivative variant of the foregoing and also combinations thereof.

In specific embodiments, chemotherapy for the individual is employed in conjunction with the disclosure, for example before, during and/or after administration of the disclosure

### Radiotherapy

Other factors that cause DNA damage and have been used extensively include what are commonly known as gamma-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells. Genes

In yet another embodiment, the secondary treatment is a gene therapy in which a therapeutic polynucleotide (e.g., a therapeutic RNA such as an mRNA or a replicon) is administered before, after, or at the same time as the present disclosure clinical embodiments. A variety of expression products are encompassed within the disclosure, including inducers of cellular proliferation, inhibitors of cellular proliferation, or regulators of programmed cell death.

Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs surgery). It is further contemplated that the present disclosure may be used in conjunction with removal of superficial cancers, precancers, or incidental amounts of normal tissue.

Upon excision of part or all the cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### Other Agents

Further agents may be used in the inventive methods. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor: F42K and other cytokine analogs: or MIP-1, MIP-1beta, MCP-1, RANTES, and other chemokines. Upregulation of cell surface receptors or their ligands such as Fas/Fas ligand, DR4 or DR5/TRAIL may potentiate the apoptotic inducing abilities of the present compositions and methods by establishing an autocrine or paracrine effect on hyperproliferative cells. Increasing intercellular signaling by elevating the number of GAP junctions may increase the anti-hyperproliferative effects on the neighboring hyperproliferative cell population. Therefore, in other embodiments, cytostatic or differentiation agents may be used in combination with the present disclosure to further enhance the anti-hyperproliferative efficacy. Inhibitors of cell adhesion may also enhance the efficacy of the present disclosure. Examples of cell adhesion inhibitors are focal adhesion kinase (FAKs) inhibitors and Lovastatin. Yet other agents that increase the sensitivity of a hyperproliferative cell to apoptosis, such as the antibody c225, may be used.

These and other aspects of the present disclosure will be further appreciated upon consideration of the following examples, which are intended to illustrate certain particular embodiments of the disclosure but are not intended to limit its scope, as defined by the claims.

### EXAMPLES

### Example 1: Effect of Inventive Cytokine Receptor Switches on CD4⁺ and CD8⁺ T Cells With and Without CD3/CD28-Costimulation

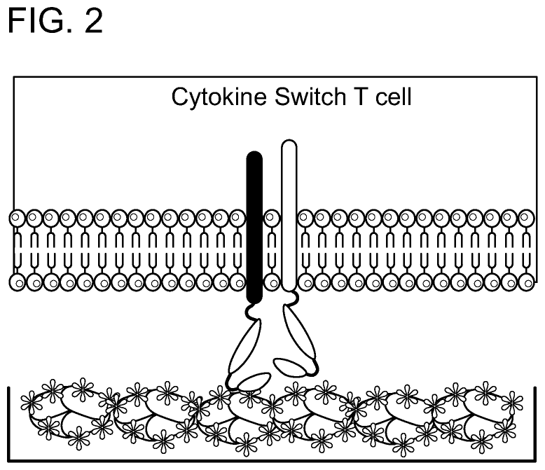

T cells comprising nucleic acids encoding cytokine receptor switches were stimulated on FITC-conjugated-BSA-coated plate (FIG. 2) with increasing dose of FITC (0, 0.1, 1, 10, 100, and 1000 μg/mL) in the presence or absence of CD3/CD28 co-stimulation for up to 2 weeks. The effector memory (CD45RA⁻ CCR7⁻), central memory (CD45RA⁻, CCR7 ⁺), and activation (CD69) markers on CD4⁺ and CD8⁺ T cells were assessed by flow cytometry.

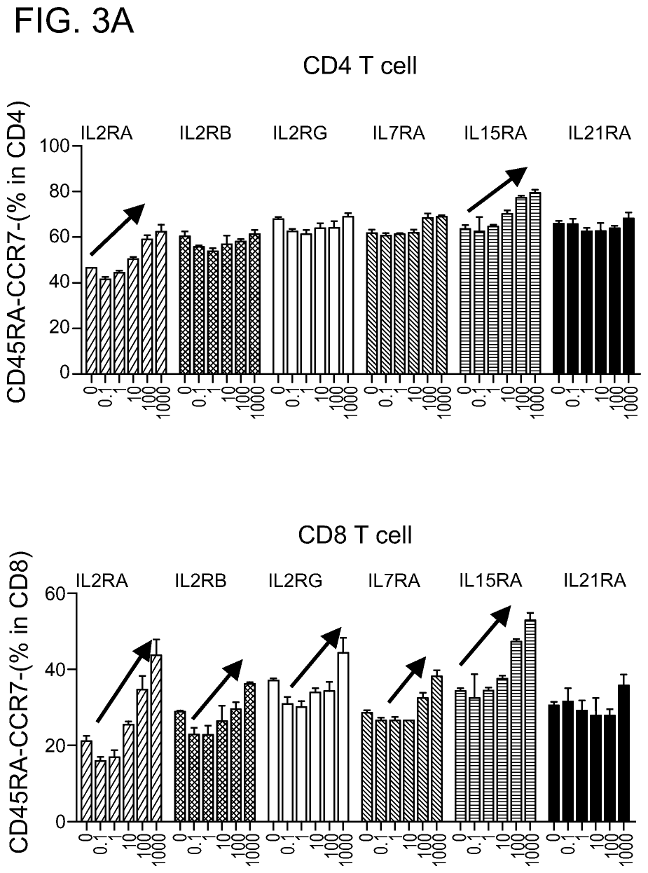

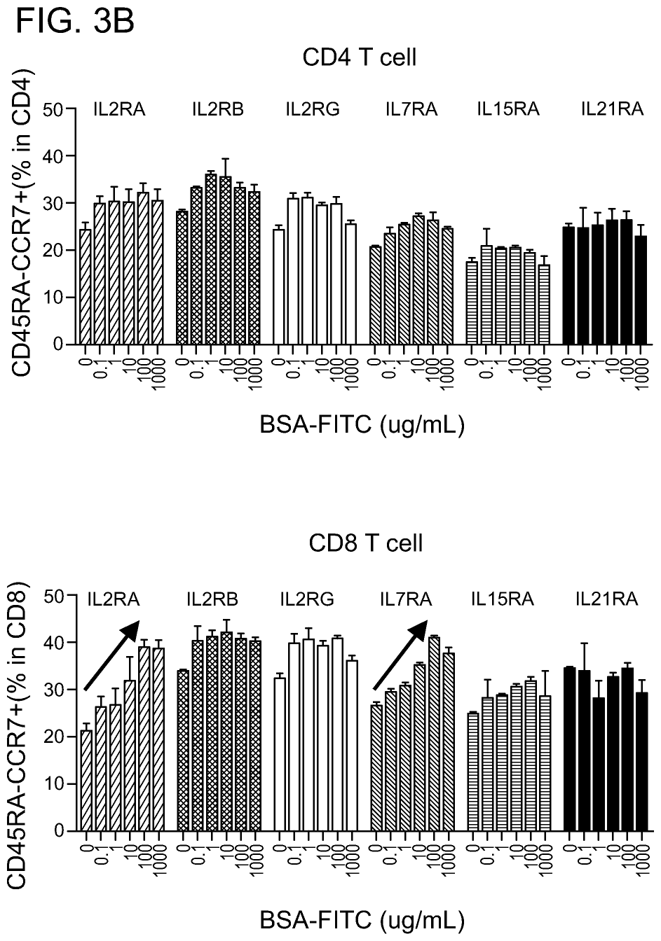

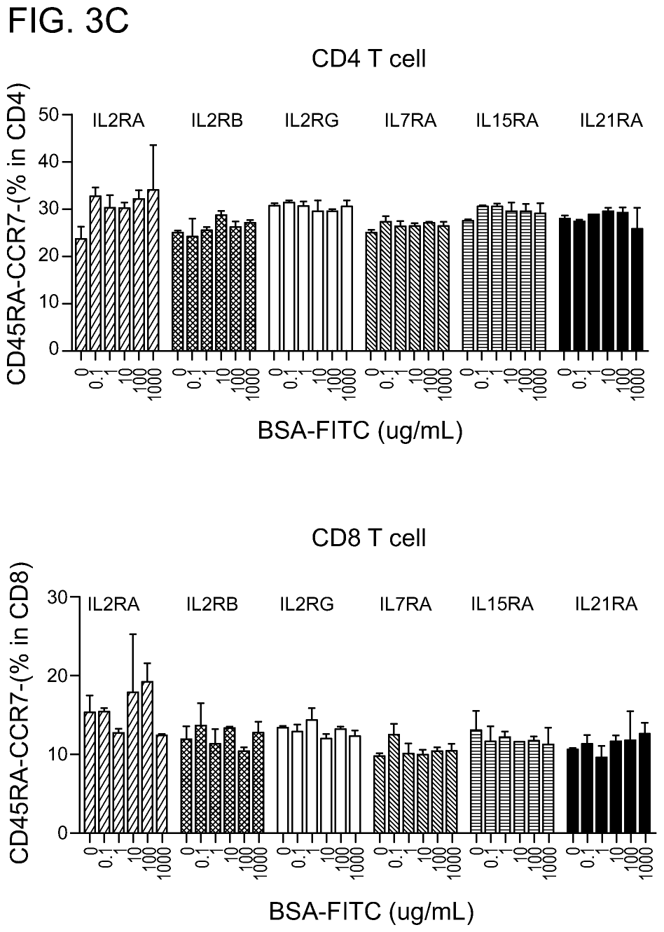

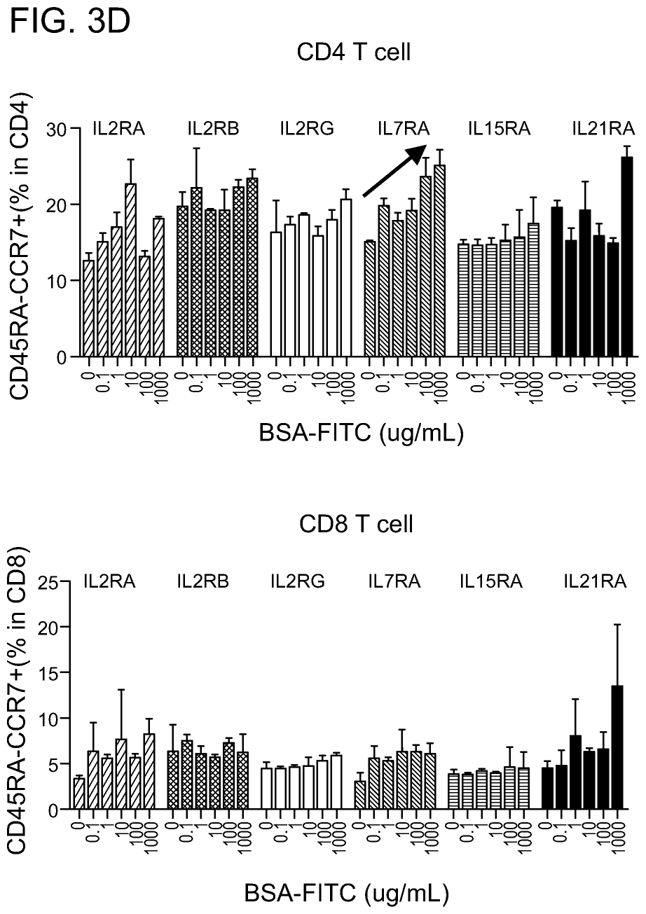

FIG. 3A shows that IL2RA-IL2RB-, IL2RG-, IL7RA-and IL15RA-cytokine receptor switches increased effector memory markers on CD8+ T cells with CD3/CD28-costimulation. IL2RA-and IL15RA-cytokine receptor switches also increased effector memory markers on CD4⁺ T cells with CD3/CD28-costimulation.

FIG. 3B shows that IL2RA-and IL7RA-cytokine receptor switches increased central memory markers on CD8⁺ T cells with CD3/CD28-costimulation.

FIG. 3C shows that cytokine receptor switches had little effect on effector memory markers on T cells without CD3/CD28-costimulation.

FIG. 3D shows that IL7RA-cytokie receptor switch increased central memory makers on CD4⁺ T cells even without CD3/CD28-costimulation.

### Example 2: BSA-FITC Bound on High-Affinity Plate Efficiently Stimulated Chimeric Antigen Receptor (CAR)-T Cells

T cells comprising a nucleic acid encoding anti-fluorescein CAR were stimulated on FITC-conjugated-antibody-coated normal (Normal) or high affinity (High affinity) plates or antibody-FITC solution (Free) with increasing doses of FITC (0, 0.1, 1, 10, and 100 μg/mL) in the presence or absence of CD3/CD28 co-stimulation. Expression of IL-2, IFN-gamma and CD69 in CD8⁺ T cells were assessed by flow cytometry.

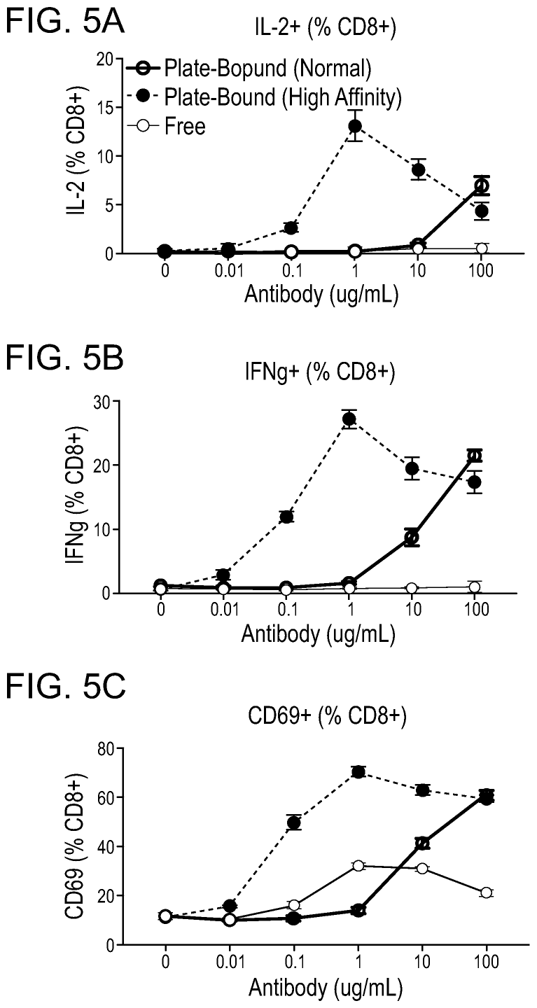

FIG. 5A shows that expression of IL-2 was increased in CAR T cells in a FITC-dose-dependent manner. Antibody-FITC bound to high-affinity plate stimulated CAR-T cells more effectively than antibody-FITC bound to normal plate or in solution.

FIG. 5B shows that expression of IFN-gamma was increased in CAR T cells in a FITC-dose-dependent manner. Antibody-FITC bound to high-affinity plate stimulated CAR-T cells more effectively than antibody-FITC bound to normal plate or in solution.

FIG. 5C shows that expression of CD69 was increased in CAR T cells in a FITC-dose-dependent manner. Antibody-FITC bound to high-affinity plate stimulated CAR-T cells more effectively than antibody-FITC bound to normal plate or in solution.

### Example 3: Effect of Inventive Cytokine Receptor Switches on NK Cells

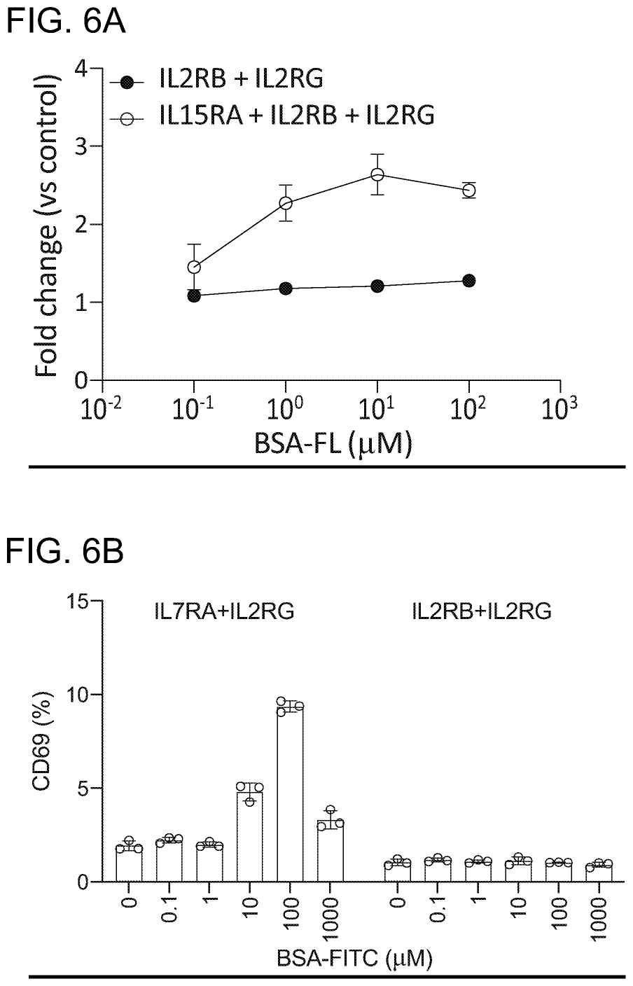

Human natural killer (NK) cell line, NK92, expressing different combinations of cytokine receptor switches (IL2RB-and IL2RG-cytokine receptor switches, or IL15RA-, IL2RB-and IL2RG-cytokine receptor switches) were stimulated on FITC-conjugated-BSA-coated plate with increasing dose of FITC (0, 0.1, 1, 10, 100 μM) for up to 7 days. The fold-increase of viable cells was assessed by cell proliferation assay. The activation marker CD69 on NK92 cells was assessed by flow cytometry. FIG. 6A shows that the combination of inventive IL15RA-, IL2RB-and IL2RG-cytokine receptor switches promoted cell proliferation of NK92 cells. These NK92 cells comprise three nucleic acids encoding three cytokine receptor switches, including a first cytokine switch comprising an IL-2RB signal peptide, an anti-small molecule scFv, a CD8 hinge, an IL-2RB transmembrane domain, and an IL-2RB intracellular domain, a second cytokine switch comprising an IL-2RG signal peptide, an anti-small molecule scFv, a CD8 hinge, an IL-2RG transmembrane domain, and an IL-2RG intracellular domain, and a third cytokine switch comprising an IL-15RA signal peptide, an anti-small molecule scFv, a CD8 hinge, an IL-15RA transmembrane domain, and an IL-15RA intracellular domain.

NK 92 cells expressing different combinations of cytokine receptor switches (IL7RA-and IL2RG-cytokine receptor switches or IL2RB-and IL2RG-cytokine receptor switches) were stimulated on FITC-conjugated-BSA-coated plate with increasing dose of FITC (0, 0.1, 1, 10, 100, 1000 μM) for up to 7 days. The activation marker CD69 on NK92 cells was assessed by flow cytometry. FIG. 6B shows that the combination of inventive IL7RA-and IL2RG-cytokine receptor switches increased the expression of activation marker CD69 on NK92 cells. These NK92 cells comprise two nucleic acids encoding two cytokine receptor switches, including a first cytokine switch comprising an IL-2RG signal peptide, an anti-small molecule scFv, a CD8 hinge, an IL-2RG transmembrane domain, an IL-2RG intracellular domain, and a second cytokine switch comprising an IL-7RA signal peptide, an anti-small molecule scFv, a CD8 hinge, an IL-7RA transmembrane domain, and an IL-7RA intracellular domain.

All patent publications and non-patent publications are indicative of the level of skill of those skilled in the art to which this disclosure pertains. All these publications (including any specific portions thereof that are referenced) are herein incorporated by reference to the same extent as if each individual publication were specifically and individually indicated as being incorporated by reference.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

What is claimed is:

1. A cytokine receptor switch comprising a signal peptide, a single chain antibody fragment (scFv) that specifically binds a synthetic, substantially nonimmunogenic small molecule (“synthetic small molecule”), a hinge domain, a transmembrane domain, and an intracellular domain native to or derived from a first cytokine receptor.

2. The cytokine receptor switch of claim 1, wherein the signal peptide is native to a second cytokine receptor, wherein the first and second cytokine receptors are the same or different.

3. The cytokine receptor switch of claim 2, wherein the signal peptide is native to IL-2RA, IL-2RB, IL-2RG, IL-4RA, IL-7RA, IL-9R, IL-15RA, or IL-21R.

4. The cytokine receptor switch of claim 3, wherein the signal peptide is native to IL-2RA and has nucleic acid sequence SEQ ID NO: 1 and amino acid sequence SEQ ID NO: 2.

5. The cytokine receptor switch of claim 3, wherein the signal peptide is native to IL-2RB and has nucleic acid sequence SEQ ID NO: 3 and amino acid sequence SEQ ID NO: 4.

6. The cytokine receptor switch of claim 3, wherein the signal peptide is native to IL-2RG and has nucleic acid sequence SEQ ID NO: 5 and amino acid sequence SEQ ID NO 6.

7. The cytokine receptor switch of claim 3, wherein the signal peptide is native to IL-4RA and has nucleic acid sequence SEQ ID NO: 7 and amino acid sequence SEQ ID NO 8.

8. The cytokine receptor switch of claim 3, wherein the signal peptide is native to IL-7RA and has nucleic acid sequence SEQ ID NO: 9 and amino acid sequence SEQ ID NO 10.

9. The cytokine receptor switch of claim 3, wherein the signal peptide is native to IL-9R and has nucleic acid sequence SEQ ID NO: 11 and amino acid sequence SEQ ID NO 12.

10. The cytokine receptor switch of claim 3, wherein the signal peptide is native to IL-15RA and has nucleic acid sequence SEQ ID NO: 13 and amino acid sequence SEQ ID NO 14.

11. The cytokine receptor switch of claim 3, wherein the signal peptide is native to IL-21R and has nucleic acid sequence SEQ ID NO: 15 and amino acid sequence SEQ ID NO 16.

12. The cytokine receptor switch of claim 1, wherein the scFv binds fluorescein or a fluorescein derivative, 4-[(6-methylpyrazin-2-yl) oxy] benzoate (MPOB), anthraquinone-2-carboxylate (AQ), or tetraxetan (DOTA).

13. The cytokine receptor switch of claim 12, wherein the scFv binds fluorescein and fluorescein derivatives and has nucleic acid sequence SEQ ID NO: 51 and amino acid sequence SEQ ID NO 52.

14. The cytokine receptor switch of claim 12, wherein the scFv binds MPOB and has nucleic acid sequence SEQ ID NO: 53 and amino acid sequence SEQ ID NO 54.

15. The cytokine receptor switch of claim 12, wherein the scFv binds AQ and has nucleic acid sequence SEQ ID NO: 55 and amino acid sequence SEQ ID NO 56.

16. The cytokine receptor switch of claim 12, wherein the scFv binds DOTA and has nucleic acid sequence SEQ ID NO: 57 and amino acid sequence SEQ ID NO 58.

17. The cytokine receptor switch of claim 1, wherein the hinge domain is derived from cluster of differentiation 8 (CD8).

18. The cytokine receptor switch of claim 17, wherein the hinge domain peptide has nucleic acid sequence SEQ ID NO: 49 and amino acid sequence SEQ ID NO 50.

19. The cytokine receptor switch of claim 1, wherein the transmembrane domain is derived from IL-2RA, IL-2RB, IL-2RG, IL-4RA, IL-7RA, IL-9RA, IL-15RA, or IL-21R.

20. The cytokine receptor switch of claim 19, wherein the transmembrane domain peptide is derived from IL-2RA and has nucleic acid sequence SEQ ID NO: 17 and amino acid sequence SEQ ID NO 18.

21. The cytokine receptor switch of claim 19, wherein the transmembrane domain peptide is derived from IL-2RB and has nucleic acid sequence SEQ ID NO: 19 and amino acid sequence SEQ ID NO 20.

22. The cytokine receptor switch of claim 19, wherein the transmembrane domain peptide is derived from IL-2RG and has nucleic acid sequence SEQ ID NO: 21 and amino acid sequence SEQ ID NO 22.

23. The cytokine receptor switch of claim 19, wherein the transmembrane domain peptide is derived from IL-4RA and has nucleic acid sequence SEQ ID NO: 23 and amino acid sequence SEQ ID NO 24.

24. The cytokine receptor switch of claim 19, wherein the transmembrane domain peptide is derived from IL-7RA and has nucleic acid sequence SEQ ID NO: 25 and amino acid sequence SEQ ID NO 26.

25. The cytokine receptor switch of claim 19, wherein the transmembrane domain peptide is derived from IL-9R and has nucleic acid sequence SEQ ID NO: 27 and amino acid sequence SEQ ID NO 28.

26. The cytokine receptor switch of claim 19, wherein the transmembrane domain peptide is derived from IL-15RA and has nucleic acid sequence SEQ ID NO: 29 and amino acid sequence SEQ ID NO 30.

27. The cytokine receptor switch of claim 19, wherein the transmembrane domain peptide is derived from IL-21R and has nucleic acid sequence SEQ ID NO: 31 and amino acid sequence SEQ ID NO 32.

28. The cytokine receptor switch of claim 1, wherein the intracellular domain is derived from IL-2RA, IL-2RB, IL-2RG, IL-4RA, IL-7RA, IL-9RA, IL-15RA, or IL-21R.

29. The cytokine receptor switch of claim 28, wherein the intracellular domain is derived from IL-2RA and has nucleic acid sequence SEQ ID NO: 33 and amino acid sequence SEQ ID NO 34.

30. The cytokine receptor switch of claim 28, wherein the intracellular domain is derived from IL-2RB and has nucleic acid sequence SEQ ID NO: 35 and amino acid sequence SEQ ID NO 36.

31. The cytokine receptor switch of claim 28, wherein the intracellular domain is derived from IL-2RG and has nucleic acid sequence SEQ ID NO: 37 and amino acid sequence SEQ ID NO 38.

32. The cytokine receptor switch of claim 28, wherein the intracellular domain is derived from IL-4RA and has nucleic acid sequence SEQ ID NO: 39 and amino acid sequence SEQ ID NO 40.

33. The cytokine receptor switch of claim 28, wherein the intracellular domain is derived from IL-7RA and has nucleic acid sequence SEQ ID NO: 41 and amino acid sequence SEQ ID NO 42.

34. The cytokine receptor switch of claim 28, wherein the intracellular domain is derived from IL-9R and has nucleic acid sequence SEQ ID NO: 43 and amino acid sequence SEQ ID NO 44.

35. The cytokine receptor switch of claim 28, wherein the intracellular domain is derived from IL-15RA and has nucleic acid sequence SEQ ID NO: 45 and amino acid sequence SEQ ID NO 46.

36. The cytokine receptor switch of claim 28, wherein the intracellular domain is derived from IL-21R and has nucleic acid sequence SEQ ID NO: 47 and amino acid sequence SEQ ID NO 48.

37. The cytokine receptor switch of claim 1, which is an anti-fluorescein-IL2-RA cytokine receptor switch and has amino acid sequence SEQ ID NO: 59.

38. A nucleic acid encoding the cytokine receptor switch of claim 1.

39. A composition comprising an immune cell containing the nucleic acid of claim 38.

40. The composition of claim 39, wherein the immune cell further comprises at least two of the nucleic acids, wherein the two nucleic acids encode different cytokine receptor switches that comprise different signal peptides, different transmembrane domains, and/or different intracellular domains of the cytokine receptors.

41. The composition of claim 40, wherein the at least two cytokine receptor switches comprise different scFvs.

42. The composition of claim 40, wherein the cytokine receptor switches comprise the same scFv.

43. The composition of claim 40, wherein the at least 2 cytokine receptor switches comprise a first cytokine receptor switch comprising an IL-2RG signal peptide, an anti-small molecule scFv, a CD8 hinge, an IL-2RG transmembrane domain, an IL-2RG intracellular domain, and a second cytokine receptor switch comprising an IL-7RA signal peptide, an anti-small molecule scFv, a CD8 hinge, an IL-7RA transmembrane domain, and an IL-7RA intracellular domain.

44. The composition of claim 40, wherein the immune cell further comprises at least a third nucleic acid that encodes a third cytokine receptor switch that differs from the first and second cytokine receptor switches.

45. The composition of claim 44, wherein the at least 3 cytokine receptor switches comprise a first cytokine receptor switch comprising an IL-2RB signal peptide, an anti-small molecule scFv, a CD8 hinge, an IL-2RB transmembrane domain, and an IL-2RB intracellular domain, a second cytokine receptor switch comprising an IL-2RG signal peptide, an anti-small molecule scFv, a CD8 hinge, an IL-2RG transmembrane domain, and an IL-2RG intracellular domain, and a third cytokine receptor switch comprising an IL-15RA signal peptide, an anti-small molecule scFv, a CD8 hinge, an IL-15RA transmembrane domain, and an IL-15RA intracellular domain.

46. The composition of any claim 39, wherein the immune cells are T cells or NK cells.

47. The composition of claim 46, wherein the T cells are CD8⁺ or CD4⁺.

48. The composition of claim 39, wherein the immune cells further comprise a nucleic acid encoding a chimeric antigen receptor (CAR) directed against a cell surface antigen.

49. The composition of claim 48, wherein the cell surface antigen is CD19, B-cell maturation antigen (BCMA), human epidermal growth factor receptor 2 (HER2), or epidermal growth factor receptor (EGFR), mucin 1 (MUC1), or TNF receptor superfamily member 13B (TNFRSF13B).

50. The composition of claim 46, wherein immune cells are binary activated T cells that encodes a chimeric antigen receptor (BAT-CAR).

51. The composition of claim 46, wherein immune cells are binary activated NK cells comprising a nucleic acid encoding a chimeric antigen receptor.

52. A method for stimulating the immune cells in the composition of claim 39, comprising contacting the immune cells with a sufficient concentration of the synthetic small molecule to promote proliferation.

53. The method of claim 52, wherein the contacting also promotes a change in phenotype of the immune cells.

54. The method of claim 53, wherein the treating promotes a change in phenotype selected from memory, cytotoxic, and regulatory phenotypes.

55. The method of claim 52, wherein the synthetic small molecule is fluorescein or a fluorescein derivative, MPOB, AQ, or DOTA.

56. The method of claim 52, wherein the synthetic small molecule is conjugated to a carrier.

57. The method of claim 56, wherein the synthetic small molecule conjugated to the carrier is bound to a high-affinity plate, dish, or flask.

58. The method of claim 56, wherein the carrier is bovine or human serum albumin, dextran, or an antibody.

59. The method of claim 52, wherein the synthetic small molecule is a polymer.

60. The method of claim 59, wherein the polymer is a monopolymer, heteropolymer, or a branched polymer.

61. The method of claim 58, wherein the antibody is an anti-HER2 antibody, anti-EGFR antibody, anti-BCMA, or anti-CD19 antibody.

62. The method of claim 61, wherein the antibody is Pertuzumab, Cetuximab, Belantamab, J6M0, or Daratuzumab.

63. The method of claim 52 or 53, wherein the concentration of the synthetic small molecule ranges from 0.1 to 1000 μg/mL, based on total volume of the composition.

64. The method of claim 63, wherein the concentration of the synthetic small molecule ranges from 0.1 to 100 μg/mL, based on total volume of the composition.

65. The method of claim 52, wherein the immune cells are treated with the sufficient concentration of the synthetic small molecule for up to two or more weeks.

66. The method of claim 52, wherein the immune cells are treated with the sufficient concentration of the synthetic small molecule for one week.

67. The method of claim 52, wherein the immune cells are contacted with the synthetic small molecule ex vivo.

68. The method of claim 52, wherein the immune cells are contacted with the synthetic small molecule in vivo.

69. The method of claim 68, wherein the immune cells are contacted with the synthetic small molecule systematically.

70. The method of claim 68, wherein the immune cells are contacted with the synthetic small molecule locally.

71. A method for treating a disease or disorder comprising:
administering to a subject in need thereof a therapeutically effective amount of the composition of claim 39; and
administering to the subject a sufficient concentration of at least one synthetic small molecule conjugated to a carrier thereby stimulating the immune cells in the composition.

72. The method of claim 71, wherein the synthetic small molecule is fluorescein or a fluorescein derivative, MPOB, AQ, or DOTA.

73. The method of claim 71, where the carrier is human serum albumin or an antibody.

74. The method of claim 73, wherein the antibody is an anti-HER2 antibody, anti-EGFR antibody, anti-BCMA antibody, or anti-CD19 antibody.

75. The method of claim 74, wherein the antibody is Pertuzumab, Cetuximab, Belentamab, J6M0, or Daratuzumab.

76. The method of claim 71, wherein the disease or disorder is cancer.

77. The method of claim 76, wherein the cancer is breast cancer, ovarian cancer, multiple myeloma, lung cancer, or glioblastoma multiforme.
